SEQUENCE LISTING (60 sequences)

SEQ ID NO: 1 - DNA, 2316 bp, Trypanosoma congolense
atgtgtgaaaggaagtcggtgtttgcattccctgaggtagtcaagaatgggcgatcagtctctggggggcatcagggaacctggtgcacgccgctgttgcttatctcagcaatatttctgccgctgacatgctgcagcgagtccacagattcaacgtggctggagaagcggagagtggagctcttcagaccctggggcaaagggaaccctaacgtaccgggcgcatcatactcctctgacgggcgcggtgtgttcgagggcaactcattgctggaggtaaatgaccagattgtgacgctcgccggcgccaggtacaattcgtgggtcgatgggtacgccggaatgtggatgaaaactatccgcctgagtgaagggcatcagggccccggagccgctgactggatgcaagaaaagaactggaagggtgaggctgttatcgtgaatgaaaaggtagagtctcatcgatatgcgttgatgggcccgagagcagcagtggtaggtgacaagatctttttcctttctatcaccagcaacaaatcaaaggatgcattgtcaagcccgtccgacgaatcaaatctagacgttaggctatacattggaactgtcgataaaagttttgttggtgatgcttctgtccactggaacggccctcgttcactccttgtgacatttatgaaggagctgaagaagaactcgtggaaggactttgtagaaggcagcggtaaaagtgttgtaatgggcgacacaatattctttccacttgttgccctaactcacaagcagagtaggtcatgcgtcattgcccgctataggcataacgatgagaattggaccttcacccgcgttgcactggatattgatgactgcacaaaccctacccttcttctgtggaaaaacgagctgatgatagtcgttgcgcacaacttgaagaacaaagtgtacagatctgtggatatggggctaacatggacggatgcctcgaagacacgcagatatgcactgactaacttccagcaccacgccgacgatgttgaccggggtgacatcttgagtgtcagggtcggtgagactgatttgcttctttttgcctatcgtatgttcttcagctccgcgacggcaggcaaccggccgcttctcctgtggatgacagacaacaagcgcacacactgcctcggacccatatcaaccggtcacctgttcacgggcgcctttggtgccctactgtatacaagggaaaagctgtattctctccatcaggaaagcttcagttccctaagcagtctcttttttaccaatctcaccggcagactgcgcacgatgcggccggtgctcgatacatggaagactgccgataaacgtgttatggggctttatggcccgtctgccgccgggacgaccaatttcaaatccgctgagccgtccagcttcgatcccacaacggggcttgttggcttttggtccaccgcgagtaatgccacgcactggcaggatgagtaccttggcatggacggtgtgctgcatggccccttgaagagggtgactacgggctacacgatggaggggtgtgccgctcatgttgtgtggcccgtggggggcgagagcgaaaacaaagtgtatcacctaataagcaacggtctcaccgtcgtcatgagtgtggctgttcacactgcccccaaggttcgcattcctctccttggagtaaccgtgaggaacggttcaaactgggccactgatgtgggtatctggtatgacaacaagacatgggcacagatgggcggtgatgaagttggcgcagtactcgctatggaggtagggaagacgtaccagctggtcttcacagtcaaaggaggcgtcgcacgcacttacgttgacgggcggcgcgtgggcgcagagaggggtatcatagtgccgcagtcgcagtccatggaagtggacgaaatgtacattggcaaccgtgacaaggctatgacaaagtgctcagcagacgccctcaacgtgactgtgttcaacatgctgctgtataactacgagctcagccccgctgacgtgaggacgttgcttaccatgaagggcaggagcgcgttcgagactatcggaatgagcggtgacgatgaagaacaggaggctgagtcgggcggcggcagtatgctgtggaccttggcagtgttaatccccgcgattgttctccttttcggtgcggctgcattcttcctagtgcggcgtcggagagctggcacaacgatgcctccagcgaccgtgcaccacaacccttactttatgaacgccacagacgatacgttggaggtgagcaagtga

SEQ ID NO: 2 - DNA, 2073 bp, Trypanosoma congolense
atgtgtactactggcatgcgagtggcactcacggtactttgtttgactcattacatccttgaacaggcgcgagttggtgtcacccctaatgcaggccatgaaccaaattccgttaatgaatttacactctttgccgaaggcgaggagcatacctataggcttgccgctgttgactccgttcacatacattctcttgtcaaagttggggacgtgctggtggctatcggagaacggaggtatcgtttggcaggtgaaatgcgactcaacactttctccctgtgcagcgtagatggtggtaagacatggacaaaggatgttatagctgtgggtatggggagtacgagttaccactcttaccccatactgtacgaggcaattgtaaaagagaatagtatttacctatttgccggaggatacgatatagatactgttgggacaggtaatattaatataagtagccgaggttgggacccccttcttatcgttggaaaggtggaagtatcgcgcggtttattttcccagtcagccaaagtaacttggggtacacaagttcccttaaaggggagcattcctgatgggctcagaatgggacctgtgagcaagttttatcggggagtaaagggtgcagttgtgacagaggtggggtcacttgtatttcttgtggagctgacaaatagccacaatcaagacgttcctgttgtgatttactctacgaatgacggcgagaattggaacctagagcctctcgatcctggtgtgtgtaaaggttattgtcacatttttgtatggaatggacgactcatgctgggtaaccaatcgagtaagggacatcaaattgtatacgagtcaattaacttcggaagggagtgggtagaggctgtcacttcctattcgcgtgtgtgggcgatcgaagcagaacacggcaagctatacaattttgttacagcaacagttgaaggaagaagggtgttggtctttgcccagcgaagcatcaatgataaattgcgagaggtgctccggatttggctctctgatggtgaccattttgctgaaatagatcatattcatctcgatgatgacatcgtcggtgagggaacgttgttgttcgatgagaacacgctcctttacttctacagaaaaataggatatttgcgcgatgaatttagcagttcagtgccgtatgacattggtaacattgcccagttggatgatgcattggcaaagataaaatctgtgctgcgtatgtggaaaattgaatcaacaggtgcagttgaaggtggcggtgtcgtcaagaatctcagatgcattgatgtctcgcctgtcgtactcctttccaacgatgttaacgccactcactggaaggatgtttacggcactgccaacattaacgtaacaggggctacaaaagccgatggtggcgttttatttagggggacgaacagaggtgctgcatggtatgtaggtgagaggagtgggacacagatgtacaccttcgtaaattacgaattcacacttgtgatgacggtggtgataagtgaaggagtgaaagagaacatccctgttttggctgtagccgtcaacgaaggtgatagcaacaaaattctcgaggtatcttacaatgccgacgggaggtggcacttaacatttggcggtaagtatgtgcctactgtcggctttcatttgcacaactcaacgcatcaagtggcagttaccatgtacggtggctctttttccgttaaggttgatggcacggcattatccagcgcgagaaatagcatcaaggttctcaaacagccctcgcgcatttcatatttttacattgggggttatggtaacccaaggaccacccctaatggggagcttatggttcggaatgtagcactctacaagcgggagctaagttccctagagctggacgtgatgtttttacaatcttattgggccaggtgccctgccaaatcgttgttggctgcacaagaaaaaccaacaggtgatggagtcgaggcgccaggccgtatgggactgtttctttacttgttgctggctataatatcttacgctgtccaggcatag

SEQ ID NO: 3 - DNA, 2097 bp, Trypanosoma congolense
atgagccccacgaggatggcgaacgttcatgcacttgcgcacgcggggatgcgactcgtctctttcgcatttgttgttctcattcgcgtgagccacatgccctccgaggggctagcgcgggtcgatgctggtgataaggggaaccatgaggtaggggtcaacagaacggggccgtactcgtataggtccccatcgctcctcgcagtgcaaggctcactgatcaccgtgtcagaaacgtgggacaccacggacgaaaagaagtatgtggatgtaatcacggaatacagccgagactatggtacgtcgctggtgacgcaggttgcaattcggagtgacaaggcggacttccatgcggtttacacacatcaggaagaccgcgagagtactcttcatccaaccgccgttgctagcggtgacaaggtgtacgtccttgtcttttgcaagaatataggtgcgaacgactcgctgactggtgatcaggtgattatgccgtatgtggccacagggaccgtgctaccgctcggtgcaattggcgaaacgtgggtggattggacggcactcaacccgattagggctctcctccctggatttgttggagggaagagggcaagtaggttttttggtggcggcggtaatggcattgcgacaccgcaaggaacgatcattatccccgtgcaggtggtgcgaacggacgatgaatactttgcctcaattatatactcaacgaacggtggctcgagttgggctctcgcgaaaggagtaacagacgcagggtgcagagaatcttcagttctggaatggaagggcaagctcctcctggtttcacggtcaaacgacggcttcacaaaagtttatgaatcgggcgacatgggcaccaagtggacagaagctctcgggacgatatcgcgcgtgttcggcaactcaccaaaccgtacaggacccggtaaccaaggcagtgccgtagttgccaatattgacaacgtccctgttatgattttttcgcacacaactgtactccacggtggcggtgacggtgatgacagtgggcgcatccgtgaaattcaccagcgcatatggcttagtgacggcaaccgcattgtgaaagtagggcacatttactgggatgaccacctgcaaagcagccacaacaaccttctctacgacaagggaaaacttttctgtgcatacgaggcgggagcggagaagacatccgcggttctcgtcaggagcctcgacgatgagctgagcaaggttgaggcagcgttggaggcatggaagaggcaagacagctacctctcaaccgtgtgtgcatcggggtcggataccgccccatgcgaaagcggggttcccatcgacggccttgtggggctcctctccaccacgctaagcgagaggcaatggatcgacgcttacttatccgtcagcgctgaggtggtgggcgcacggagcattccccaaggggtgttgtttgagggacctattcgtgggggcaggtggccagtcgccgcacaggggcaaaatcagcggtaccactttgtcagcaaacactttacgctggtgatcacggtgagcatacacgagcgaacaacagacagggcgccgctgctggtattgcggccccaagaggatgccggcgcagacttggaactttcgtacacagccgaccatcggtggcacgtgcggcacggaaacgaacatgggtccacgtcgggggcgtgggtgaaggaccgggagcaccagttggtgctcgtgtgcgaggcgggggatgcctcgctctatcttgacgggaagcgcatgccgacgatgggaagaaggctggtggagagcggagcgccacttggagtatcccacttcagcataggtggatatggactcgagaagaggagccccaacggtaagctgaccgtgaggaatgttatgttatataatcgcccactaaataagacggagattgacacagtgttccatgtgagggataaaatcaccgccgccacgactatagtaaaggcatttgaacaaaaaaaccgcgtcaacgtccagatggtgaacagcaaacaggaccaccccaccgctcctaacaatgaggaggcatgtggg

SEQ ID NO: 4 - protein, 771 aa, Trypanosoma congolense
MCERKSVFAFPEVVKNGRSVSGGHQGTWCTPLLLISAIFLPLTCCSESTDSTWLEKRRVELFRPWGKGNPNVPGASYSSDGRGVFEGNSLLEVNDQIVTLAGARYNSWVDGYAGMWMKTIRLSEGHQGPGAADWMQEKNWKGEAVIVNEKVESHRYALMGPRAAVVGDKIFFLSITSNKSKDALSSPSDESNLDVRLYIGTVDKSFVGDASVHWNGPRSLLVTFMKELKKNSWKDFVEGSGKSVVMGDTIFFPLVALTHKQSRSCVIARYRHNDENWTFTRVALDIDDCTNPTLLLWKNELMIVVAHNLKNKVYRSVDMGLTWTDASKTRRYALTNFQHHADDVDRGDILSVRVGETDLLLFAYRMFFSSATAGNRPLLLWMTDNKRTHCLGPISTGHLFTGAFGALLYTREKLYSLHQESFSSLSSLFFTNLTGRLRTMRPVLDTWKTADKRVMGLYGPSAAGTTNFKSAEPSSFDPTTGLVGFWSTASNATHWQDEYLGMDGVLHGPLKRVTTGYTMEGCAAHVVWPVGGESENKVYHLISNGLTVVMSVAVHTAPKVRIPLLGVTVRNGSNWATDVGIWYDNKTWAQMGGDEVGAVLAMEVGKTYQLVFTVKGGVARTYVDGRRVGAERGIIVPQSQSMEVDEMYIGNRDKAMTKCSADALNVTVFNMLLYNYELSPADVRTLLTMKGRSAFETIGMSGDDEEQEAESGGGSMLWTLAVLIPAIVLLFGAAAFFLVRRRRAGTTMPPATVHHNPYFMNATDDTLEVSK

SEQ ID NO: 5 - protein, 699 aa, Trypanosoma congolense
MCTTGMRVALTVLCLTHYILEQARVGVTPNAGHEPNSVNEFTLFAEGEEHTYRLAAVDSVHIHSLVKVGDVLVAIGERRYRLAGEMRLNTFSLCSVDGGKTWTKDVIAVGMGSTSYHSYPILYEAIVKENSIYLFAGGYDIDTVGTGNINISSRGWDPLLIVGKVEVSRGLFSQSAKVTWGTQVPLKGSIPDGLRMGPVSKFYRGVKGAVVTEVGSLVFLVELTNSHNQDVPVVIYSTNDGENWNLEPLDPGVCKGYCHIFVWNGRLMLGNQSSKGHQIVYESINFGREWVEAVTSYSRVWAIEAEHGKLYNFVTATVEGRRVLVFAQRSINDKLREVLRIWLSDGDHFAEIDHIHLDDDIVGEGTLLFDENTLLYFYRKIGYLRDEFSSSVPYDIGNIAQLDDALAKIKSVLRMWKIESTGAVEGGGVVKNLRCIDVSPVVLLSNDVNATHWKDVYGTANINVTGATKADGGVLFRGTNRGAAWYVGERSGTQMYTFVNYEFTLVMTVVISEGVKENIPVLAVAVNEGDSNKILEVSYNADGRWHLTFGGKYVPTVGFHLHNSTHQVAVTMYGGSFSVKVDGTALSSARNSIKVLKQPSRISYFYIGGYGNPRTTPNGELMVRNVALYKRELSSLELDVMFLQSYWARCPAKSLLAAQEKPTGDGVEAPGRMGLFLYLLLAIISYAVQAGGGTDNRVA

SEQ ID NO: 6 - protein, 717 aa, Trypanosoma congolense
MSPTRMANVHALAHAGMRLVSFAFVVLIRVSHMPSEGLARVDAGDKGNHEVGVNRTGPYSYRSPSLLAVQGSLITVSETWDTTDEKKYVDVITEYSRDYGTSLVTQVAIRSDKADFHAVYTHQEDRESTLHPTAVASGDKVYVLVFCKNIGANDSLTGDQVIMPYVATGTVLPLGAIGETWVDWTALNPIRALLPGFVGGKRASRFFGGGGNGIATPQGTIIIPVQVVRTDDEYFASIIYSTNGGSSWALAKGVTDAGCRESSVLEWKGKLLLVSRSNDGFTKVYESGDMGTKWTEALGTISRVFGNSPNRTGPGNQGSAVVANIDNVPVMIFSHTTVLHGGGDGDDSGRIREIHQRIWLSDGNRIVKVGHIYWDDHLQSSHNNLLYDKGKLFCAYEAGAEKTSAVLVRSLDDELSKVEAALEAWKRQDSYLSTVCASGSDTAPCESGVPIDGLVGLLSTTLSERQWIDAYLSVSAEVVGARSIPQGVLFEGPIRGGRWPVAAQGQNQRYHFVSKHFTLVITVSIHERTTDRAPLLVLRPQEDAGADLELSYTADHRWHVRHGNEHGSTSGAWVKDREHQLVLVCEAGDASLYLDGKRMPTMGRRLVESGAPLGVSHFSIGGYGLEKRSPNGKLTVRNVMLYNRPLNKTEIDTVFHVRDKITAATTIVKAFEQKNRVNVQMVNSKQDHPTAPNNEEACGALSTSLAALLLLALLTLT

SEQ ID NO: 7 - DNA, 2205 bp, Trypanosoma congolense
atgtggccggtgaattgttacgcgctgctggcgcttgttgtggcgggtcagtgctgcggccccatgcatgccactgccgctgtgggaacgacgcaccaggcgctgctatggggctccaaatgggctctgaggaacaagacgaccccgaaagatggcgaggtgtggtggagcaacccccagccgggctggaaggaggtgtacgacgatgagtgggaggagtggtttatggagcaggagggaccaacgggtgtggatggtgtgcgtgctgagtggtaccgtcgcatgaaggatgggtatatactcgttggcggaccgaagctgaactcacccgacatgaacagcaccggcatgacgatgcggactgtgcactcgtaccgcataccctcaattgttgaggttggcggtgtgctaatgtgtgttggggatgcgcgctacatcacgtcgacggattatttcttcacggacaccgttgccgcatacagcactgacggtgggagaacgtggaagagggaggttataatcccgaatggtcgtgtggatgcccactactcccgcgtcgttgatcccactgttgttgcgaagggtaataacatttatgttctcgttgggcggtacaatgtcacgcggggctactggcacaatcagaacgacgaggctgcgatagccgattgggagcccttcgtgtacaagggcacggtgaacgtgggcacgaaggacaatgccactgatgtgtcgatcagctgggagaggactgcactgaagtcgctgtacaacttcccggtttcgggaagccctggcacgcagttccttggaggggctgggggtggtgttgtaacatccaacgggacgattgtgctgccagtgcaggcaaggaacaaagccaaccgtgttgtgagcatgatcctgtactcggctgacgatggaaagtcatggcactttgggaagggtgaggccggtgtaggcacgtccgaggctgccctcactgagtgggacggcaagctgctgattagtgcacgatccgatggtggacagggctaccgtatgatattcgaatcgagtgaccttggtgcgacgtggaaagagatgctcaacagcatctcccgcgtgattggcaactctccgggtcgcagtggtcctggcagctcgagtggcttcatcacggtgacagtggagggtgtgcctgtgatgcttctcacccatccgaagaaccttaagggctcgtattatcgtgatcgcctgcagatgtggatgacggacggcaatcgtatgtggcatgtcgggcaggtctctgagggcgacgacaacagtgcctacagctccctgctgtacactccggacggggtcctgtactgcttgcatgagcagaacattgatgaggtgtacagcctccaccttgtgcgccttgtggacgagctgaaaagcattaaatcaacggctctggtgtggaaggcacaggacgagcttctcctgggcaactgcctcccgggcgataaatatgatcccgggtgtgacggcatccccaccgctgggcttgccgggctgctggtaggacccctgacggagaagacgtggcccgacgcgtatcggtgcgtgaacgctgcaaccagcggcgctgtgagcactgctgaaggcgtgcggctcgacgtgagtggcggtggccatgttgtgtggcccgtgagtgagcaggggcaggaccagcggtattactttaccaacagcgagttcacgctcgccgtcacggtgcggtttgacgagatgccacggggggagctcccgttgctggggtttgtgaaccgcaaagggaaggtgaacaagatactgaaggtgtcgctgagcggggtggagtggctcctggcctacgggaatgagtacaacagcacagccgctgagccgctgagcgtgaacgagagccaccaggtggtgctaacgctccacgacgggatcgtctccctgcacgttgacgggggtaacacgacggcgactgtgagcgtgcgcgtggcgagccctgaggagctgctgaacattcatcatctcttcgttggcaccccagttgatggaggcgccaaggagcacgtcaacatcacagtgagcaatgtcctggtgtacaatcgaccgctgcgtggcgtggagctgcttgggctattcgcgaacaggggacggattcgtgtgcctgggagcgacaacggtgtcctcagtggcgggtaa

SEQ ID NO: 8 - DNA, 2205 bp, Trypanosoma congolense
atgtggccggtgaattgttacgcgctgctggcgcttgttgtggcgggtcagtgctgcggccccatgcatgccactgccgctgtgggaacgacgcaccaggcgctgctgtggggctccaaatgggctctgaggaacaagacgaccccgaaagacggcgaggtgtggtggagcaacccccagccgggctggaaggaggtgtacgacgatgagtgggaggagtggtttatggagcaggagggaccaacgggtgtgaatggtgtgcggggtgagtggtaccgtcgcatgacggatgggtatatactcgttggcggaccgaagctgaactcacccgacatgaacagcaccggcacgacgatgcggactgtgcactcgtaccgcataccctcaattgttgaggttggcggtgtgctaatgtgtgttggggatgcgcgctacatcacgtcgacggattatttcttcacggacaccgttgccgcatacagcactgacggtgggagaacgtggaagagggaggttataatcccgaatggtcgtgtggatgcccactactcccgcgtcgttgatcccactgttgttgcgaagggtaataacatttatgttctcgttgggcggtacaatgtcacgcggggctactggcacaataggaacgacaaggctgccatagccgattgggagcccttcgtgtacaagggcacggtgaacgtgggcacgaagggcactgccactgatgtgtcgatcagctgggagaggactgcactgaagtcgctgtacaacttcccggtttcgggaagccctggcacgcagttccttggaggggctgggggtggtgttgtaacatccaacgggacgattgtgctgccagtgcaggcaaggaacaaggccaaccgtgttgtgagcatgatcctgtactcggctgacgatggaaagtcatggcactttgggaagggtgaggccggtgtaggcacgtccgaggctgccctcactgagtgggacggcaagctgctgattagtacacgatccgatggtggacagggctaccgcatgatattcgaatcgagtgaccttggtgcgacgtggaaagagatgctcaacagcatctcccgcgtgattggcaactcgccgggtcgcagtggtcctggcagctcgagtggcttcatcacggtgacagtggagggtgtgcctgtgatgcttctcacccatccgaagaaccttaagggcgtgtactctcgtgatcgcctgcagatgtggatgacggacggcaatcgtatgtggcatgtcgggcaggtctctgagggcgacgacaacagtgcctacagctccctgctgtacactccggacggggtcctgtactgcttgcatgagcagaacattgatgaggtgtacagcctccaccttgtgcgccttgtggacgagctgaaaagcattaaatcaacggctctggtgtggaaggcacaggacgagcttctcctgggcaactgcctcccgggcgataaatatgatcccgggtgtgacggcatccccaccgctgggcttgccgggctgctggtaggacccctgacggagaagacgtggcccgacgcgtatcggtgcgtgaacgctgcaaccagcggcgctgtgagcactgctgaaggcgtgcggctcgacgtgggtggcggtggccatgttgtgtggcccgtgagtgagcaggggcaggaccagcggtattactttaccaacagcgagttcacgctcgccgtcacggtgcggtttgacgagatgccacggggggagctcccgttgctggggtttgtgaaccgcaaagggaaggtgaagaagatactgaaggtgtcgctgagcggggtggagtggctcctggcatacgggaatgagtacaacagcacagccgctgagccgctgaacgtgaatgagagccaccaggtggtgctaacgcttcacgacgggatcgtctccctgcacgttgacgggggtaacatgacggcgactgtgagcgtgcgcgtggcgagccctgcggagctgctgaacattcatcatctcttcgttggcaccccagttgatggaggcgccaaggagcacgccaacatcacagtgagcaatgtcctggtgtacaatcgaccgctgcgtggcgtggagctgcttgggctattcgcgaacaggggacggattcgtgtgcctgggagcgacaacagtgtcctcagtggcgggtaa

SEQ ID NO: 9 - DNA, 2202 bp, Trypanosoma congolense
atgcggccggtgaattgttacgcgctgctggcgcttgttgtggcgggtcagtgctgcggccacatgcatgccactgccgctgtgggaacgacgcaccaggcgctgctgtggggctccaaatgggctctgaggaacaagacgaccccgaaagatggcgaggtgtggtggagcaacccccagccgggctggaaggaggtgtacgacgatgagtgggaggagtggtttatggagcaggagggaccaacgggtgtgaatggtgtgcggggtgagtggtaccgtcgcatgaaggatgggtatatactcgttggcggaccgaagctgaactcacccgacatgaacagcaccggcacgacgatgcggactgtgcactcgtaccgcataccctcaattgttgaggttggcggtgtgctaatgtgtgtgggggatgcgcgctacatcacgtcgacggattatttcttcacggacaccgttgccgcatacagcactgacggtgggaaaacgtggaagacggaggttataatcccgaatggtcgtgtggatgcccactactcccgcgtcgttgatcccactgttgttgcgaagggtaataacatttatgttctcgttgggcggtacaatgtcacgcggggctactggcacaatcagaacgacgaggctgccatagccgattgggagcccttcgtgtacaagggcacggtgaacgtgggcacgaagggcactgccactgatgtgtcgatcagctgggagaggactgcactgaagtcgctgtacaacttcccggtctcgggaagccctggcacgcagttccttggaggggctgggggtggtgttgtaacatccaacgggacgattgtgctgccagtgcaggcaaggaacaaagccaaccgtgttgtgagcatgatcctgtactcggctgacgatggaaagtcatggcactttggggagggtgaggccggtgtaggcacgtccgaggctgccctcactgagtgggacggcaagctgctgattagtacacgatccgatggtggacagggctaccgcatgatattcgaatcgagtgaccttggtgcgacgtggaaagagatgctcaacagcatctcccgcgtgattggcaactcgccgaaacgtaatggtcctggcagctcgagtggcttcatcacggtgacagtggagggtgtgcctgtgatgcttctcacccatccgaagaaccttaagggctcgtattatcgtgatcgcctgcagatgtggatgacggacggcaaccgtatgtggcatgtcgggcaggtctctgagggcgacgacaacagtgcctacagctccctgctgtacactccggacggggtcctgtactgcttgcatgagcagaacattgatgaggtgtacagcctccaccttgtgcgccttgtggacgagctgaaaagcattaaatcaacggctctggtgtggaaggcacaggacgagcttctcctgggcaactgcctcccgggcgataaatatgatcccgggtgtgacggcatccccaccgctgggcttgccgggctgctggtaggacccctgacggagaagacgtggcccgacgcgtatcggtgcgtgaacgctgcaaccagcggcgctgtgagcactgctgaaggcgtgcggctggacgtgggtggcggtggccatgttgtgtggcccgtgagtgagcaggggcaggaccagcggtattactttaccaacagcgagttcacgctcgccgtcacggtgcggtttgacgagatgccacggggggagctcccgttgctggggtttgtgaaccacaaagggaaggtgaacaagatactgaaggtgtcgctgagcggggtggagtggcttctggcctacgggaatgagtacaacagcacagccgctgagccgctgagcgtgaacgagagccaccaggtggtgctaacgctccacgacgggatcgtctccctgcacgttgacgggggtaacacgacggcgactgtgagcgtgcgcgtggcgagccctgaggagctgctgaacattcatcatctcttcgttggcaccccagttgatggaggcgccaaggagcacgccaacatcacagtgagcaatgtcctggtgtacaatcgaccgctgcgtggcgtggagctgcttgggctattcgcgaacaggggacggattcgtgtgcctgggagcgacaacggtgtcctcagtggcggg

SEQ ID NO: 10 - DNA, 2337 bp, Trypanosoma congolense
atgagccattgcttcgtgccagcatggtccaaggccttgtgcctcctgctgctttctcactgcctttacttggcgcacgcctctggaaacggacgaacgacacgtgagttgtttctgggagggggccattgggtcgtgggtaaggagtgccttgccgttaacgaagaaggttccgccgcccggactctggaatgcaatgggaactgcagccccgatgaagactcacagagacgaagtgcggatgacaatgatggcttacaggaggagaccattaattgcgtactagagcccagatcgaagcaacttggtgttgctaaagatatggaagggaagcatgttgtggactcatttcgtattccatcgatcgttgaagtggacggtgtattaattaccgtatctgacgtgcgttacctcaactctaacgacctttcttttattgacacggttgccagatacagtgcagatggtgggaggacgtgggagactgaagtaataatcaagaatgctagagtgaatgcagagcactctcgcgtcgttgatcccactgttgttgtcaaaaacaacaccatatttgttctcgttgggaggtacaacaagagtgatgcgtactggacgtggcagggaggtggtggcgattgggatattctcatgcacaagggcactgtgacgaagtcgctgagaggtgggaaaccatctgtaaacattgagtgggatgagccacaaaacttgaagtatttgctgagcacagttggtaagatagatggtaggtcactgattcagtatattggcggagttggcaactgtgtcgtaacgccgaacggtaccatcgtactccctgttcaagtcctgaacacgaacagatctgtcatggccatgataatttactcaacagatgaaggggaatcgtggcagtttagcaagagtgctacaccggttgggacaacagagtcctctattgtgtggtgggatgacaaactgctcctgaatggtagaacaaacaatgatttaggctaccgcaaggtttacgaatcaagcgaccttggcacaacatggacagaagtcgttgggaccatttcacgcgtgatcggaaactcgccgggccgcaatcaaccgggaagttcagggagttctattgccataacattggaagggatgcgtgtgatgctcataactcaaccgaagaatataaaaggttcttggcaccgtgaccgtttgcagctgtggttgacggacggtaatcgcgtgtggctggtggggcagatttccgaaggtgatgataacgggccctacagctccctgttgtatacgtcgaatgggaccctctactgcttgtacgagcaggacaaagccgccgttttgagtatctaccttatcaagctagaagacgagctggaaagcattaagtccatagtgaagctatggaaggatcaggacgcgcttctctcagggaactgttcctcaccggatggtgattatactgagggctgcgtcggcattcccactgctggcctcgttgggctgctctctggaccttcagatgaggatgtatggcatgacgcgtaccgttgtgtcgatgccagtgtcgaaaatgtggtcaatgttgcagatggcctgcagctgagtggttggaacagcagccgtgtgctttggcctgtcagcagtcaggggcaggaccaaaaataccactttgccgatgttcacttcacgttggttgcaaatctgcgtctggtaggggcgccaaagggcgacttctcactggtggggtttgagatgtacgaaggagagacgaggaaaactgtaaaactgtcggctataaagagcgccttatgggagatgtgtcacaccgatttaaccacgaggggatccaggggctctcttccctgcgacgaagttcatcaagtcgcactcacacttcgtaatggtgtgatatctgtttatgccaacggaagacacctatcagtgctggatacgaaggttgcgggcgccaatgagcttctaaatatatctaacttctttgttgggcatccgggagtggggggtgctttgccgtggggcagcgcagtggtacgagatgtgctgctctataaccgcccgctgcatgagactgaactggagtcactttacctcaacggggatgtaataaaagtggtgaaccacggcgcagctggcatatccgctgctcgtgatgctgaactgctgcatgtccgaggagatggtggagacaagcccgatgctgtaccattgaagctagcaataattaccggtgatggtgtggtgcggttcagagggctttaccaaatggcttctctcgtgctacttggattaatgttatcttaa

SEQ ID NO: 11 - DNA, 2337 bp, Trypanosoma congolense
atgagccattgcttcgtgccagtatggtccaaggccttgtgccttctgctgctttctcactgcctttacttggcgcacgcctctggaaacggacgaacgacacgtgagttgtttctgggagggggccattgggtcgtgggtaaggagtgccttgctgttaacaaggaaggttccgtcgcccagactctggaatgcaatgggaactgcagccccgatgaagactcacggatacgaagtgcggatgacaatgatggcttacaggaggagaccattaattgcgcactagagcccagatcggagcaacttggtgttgccaaagatatggaagggaagcatattgtggactcatttcgtattccatcgatcgttgaagtggacggtgtattaattaccgtatctgacgtgcgttacctcaactctaacgacctttcttttattgacacggttgccagatacagtgcggatggtgggaggacgtgggagactgaagtaatcatcaagaacgccagagtgaatgcagagcactctcgtgtcgttgatcccactgttgttgtcaaaaacaacaccatatttgttctcgttgggaggtacaacaagagtgatgcgtactggacgtggcagggaggtggtggcgattgggatattctcatgcacaagggcactgtgacgaagtcgctgagaggcgggaaaccatctgtaaacattgagtgggatgagccacaaaacttgaagtatttgctgagcacagttggtaagatagatggtaggtcactgattcagtatattggcggagttggaaactgcgtcgtaacgccgaacggtaccatcgtactccctgttcaagtcctgaacacgaacagatctgtgatggccatgataatttactcaacagatgaaggggaatcgtggcagttcagcaagagtgctacaccggttgggacaacagagtcctctattgtgtggtgggatgacaaactgctcctgaatggtagaacaaacaacgatttaggctaccgcaaggtgtacgaatcgagcgaccttggcacaacatggaaagaagtcgttgggacgatttcacgcgtgatcggaaactcgccgggccgcaatcaaccgggaagttcagggagttctattgccataacattggaagggatgcgtgtgatgctaataactcaaccgaagaatataaaaggttcttggcaccgtgaccgtttgcagctgtggttgacggacggtaatcgcgtgtggctggtggggcagatttccgaaggtgatgatgacggaccctacagctccctgctgtatacgtcgaatgggaccctctactgcttgtacgagcaggacaaatccgccgttttgagtatttaccttataaagctagaagacgagctggaaagcattaagtccatagtgaagctatggaaggatcaggacgcgcttctctcagggaactgttcctcaccggatggtgattatactgagggctgcgtcggcattcccactgctggcctcgttgggctgctctctggaccttcagatggggatgtgtggcacgacgcgtaccgttgtgtcgatgccagtgtcgataatgtggtcaatgttgcagatggcctgcaactgagtggttggaacagcagccgtgtgctttggcccgtcagcagtcaggggcaggaccaaaaataccactttgccaatgttcacttcacgctggttgcaaatctgcgactggtaggggcgccaaagggcgacttctcactggtggggtttgagacgtacgaaggagagaggaggaaaactgtaaaactgtcggctataaagagcgccttttgggagatgtgtcacaccgatttaaccacgaggggatccaggggctctcctccctgcgacgaagttcatcaagtcgcactcacacttcgtgatggtgtgatatctgtttatgccaacggaagacacctatcagtgctggatacgaagattgcgggcgccaatgagcttctaaacataactaatttctttgttgggcatccgggagtgggggacgctttgccgtggggcagcgcagtggtacgagatgtgctgctctataaccgcccgctgcatgagactgaactggagtcactttacctcaacggggatgtaataaaagtggtgaaccacggcgcagctggcatatccgccgctcgtgatgctgaactgctgcatgtccgagaagacggaggagacaagcccaatgctgtcccattgaagctagcaataattaccgatgatggtgtggcgcggttcagagggctttaccaaatggcttctctcatgctacttggattaatgttatcttaa

SEQ ID NO: 12 - DNA, 2217 bp, Trypanosoma congolense
atgccctcatacctcctgcctgcagccgtagtgctgtactgctttggcggactcgttcctgctgcccagtgcatctcaacgacccgcgaggcattcatggaggccggtcagtggtcggtaaataaagattgtctcatcactgcggagggtagtcgacgttctaaggcttcaggctcttacgaaaagggatacgcttccgtcgaaagtacaacaaaagatgtgaacactcgaggtggtgtacagacaagcgaggcctgcacgttggaacccgaggtgcgcgacaactctacttcaggtgatgggaaggaaagacatttgattcactccttccgaattccctcacttgttgagatagacggcgtgcttatcgcgacattcgatacacgttatcttcacgcttccgacagcagtctcatagacacagctatgaaatacagtgccgatcaggggaagacgtggaaaactgaaatcataataaaaaatgctagactaactgataacttttcccgcgtcgttgatccaacggttgttgttaagggtgataacttgtttatttttgttgggaggtacaacacctcatctaccccatgggtctggcagaaaaacggcaaagactgggatgtactgttgtacaaggccaaggtaaggaaggaatcagcgggtggggtaccatcagtgagctttacatgggacgaacccctacacctgaagcatctgctcacctctgtcggtaaaatagacggcaggtccctcatacaatacattggtggcgttggaaatggtattgtaacaccgaaaggtactatcgtgtttccagttcaggttttaaacaccaacaaatccgtcatgaacatgcttctgtattcaagtaacgacggaaaaacctgggagttcagcaaaacttccacacccgcgggcacaactgaggcctcccttgtttggtgggatggacaactacttctcacaagcagaacaactccggatgtcggcagccgcaaagtatatttaacaagcgacctcggaacttcatggaatgaagcgatcggaagtatctctcgtgtaattggtaactcgcggtaccgtaacgatcctggggggtcaggtagctcaattgccataactgtggagggagtaccggtgatgctcgttactcacccggaaaatgcgaagggtaggtggaatcgcgaccgaatgcgactgtggctcacggacggcaatcggatgtggcttgttggacagatttctgaaggggacgacaacagtgcctacagctacctgttatacactaaaaatgggacgttattgtgtctctacgagcgaaacattcgtgagatatacagcatttatctagcccggctggaggatgagatggaggatataaagtcaactgtgaggctatggaaagcgcatgacgagctcctgtcgggagactgtcaactgaacaaaaagcgccggagcggctgcacaggcattcctatcaccggcctcgttggacttctcgcgggccttccgaggaaaagtgtttggccagatgcatataattgtgtggatgccagtatatccaagaataacaagcaagtctcccacgatccgccatcgagaagcaccatgaagcggcgtgtggtgtggccagtgggtgatcaaggtcaagaccaacgataccactttgtcaacacccactttaccttcgtcgcaaccatctattttgatcgagcgccacaggaggtttcactgatgggattcgaaaacaatgaagaaagtactaaaactctgacagtttccatcgggaacggaaggttggtcttgacgtatggaggtctgctcgaggaaatccctatgacgcggctggactggagtgtaacacatcaggtcgcgctgacacttcataatggggaggtgagcctgcacgttgatgggaacccttcgattgcgaacgtaagactgaagctccacgagcctgacaggctactaaacatctccaacttgtttacttcaaccccggcccccgtcaaaaccggtaaaggtagcacggttaccgtgaataatgtcatcctgtacaaccgtatgctaaacgaaaccgaacttgcacgtttattcaacagcagggacctcatcgatgaagtgggagatgtgcatcccgtgtctggaggtggagtcggcgagtggcgtttccacgtgtggatactactggccgcgtatgtgctcgtggcctattga

SEQ ID NO: 13 - DNA, 2364 bp, Trypanosoma congolense
atgggatattctaaatcggttcggcagacgttgatatgcctcttgttggtcgcgattgacacgtatcactgcacaacggcgtatggatcgggaatcaggggggaggaggagaagaatcgaagcttgttcctcccaggggggctttggtacaagaaagatgagtggaaggatggcaattggctgcaatctaaagagtggaaagccgggtatgcgtggtggccgtggcggtcgtggtgctccaataaaactatcggtgcgtccgggaaagaactttgccgcaaggaatgggactctcagagggaaaaaggatacacactcaagccaagggaaagcgtgctctttagggagagtagtggcacaaagagaatgcgccgcgttcactcctttcgcataccttcgatggttgaggcgaacggtgtgttaattggcattgcagacgcgcgatatttgagctccgcggacttcactttcatcgacacggttgccaagtacagtgctgacggcggtgagacgtggaaaactgaagtcatcattgaaaatgctcgtgtggattcgtttcactcccgcgtggtggatcccacagttgccgtgaagaataacagtatttatgtgcttgttggaaggtacaacacctccaacacgtattggaccatgcaaaataacggcaacgattgggacatacttatgtacaagggtaccgtaaccaagacctccgaagatggcaaacctgccgcaaacattgagtggacaggcacccagaatttgaagtacctcctgaagctggtggatcaaatagagggcaagtcgctcacgcagtttcttggtggtgtaggtaatactgtcgtaacaccggacggcacgatcgtatttcccatccaggtaaaaaattcatggaatcaagtcgccgcaatgataatgtattcgagtgacgatggcgccacgtggcacttgggaggaggcgcaacacccgttggtacaacagaggcatccgctatctggtgggacggcaaactggttctcaattgcagaacggacttggggtaccgcaaagtattcgagacaaccgaccttgggacaacgtggaaagagtcattaggggcgctctctcgtgtgattggtaactcgccagaccgcaagcagaaaggtagctcaggcagtgctattactttggaggtcgagggtgtgcaagtcatgcttattacgcagcctaagaatacgaaaggggattacaaccgtgatcgcctacaactgtggctgagtgatggcagtcgtgtgtggttagtcggacagatatcacgaggcgatgacaacagtccctacagctcgctgctgtacactagtgacgataagctttattgcctgtatgaacaggatatccggggagttttgagcatttaccttgtccatctcgttgacgaactggagaaaatcaaagcgaccgtgagattatggaaggagcaggatgcactcctctctggcaattgctctgcaactgccgaggatggcagcgattgcaacggcgtgcccaccgctggcctcgtcgggctgctttccggacctgcgcaaggaaacgcgtggcctgatgcatataattgtgtaaatgccagcctcgtgaatgttacaagtgatgcagatggtctgcagcttggcgggttgaacaggggccgtgtgtcgtggccggtgcgtgcacagggccaggatcagcggtattactttgcgaacgttcggttcactctggtcgcaactgtgcgattaaatggcatctcgaacctagaaataccgctaatgggatttgagaactttcagaaaagcacaagggataccctgattgtctcgattgtggatgatgcttactggtcgaagtgtaaggaagggccagtaccaggtgtcaatgtggatgctccggaatgccagaagtttcatcaggttgcaattatgttccaaaatggcagggtctctgtttatgccgacgggattcacatcccacagttggatacaagcatcgtggatgcctccgctctactgaatatctctagctttttccttggacatcctgaggttggcagcaggtttacttcggccgatgtaatcgtaaaaaatgtcctcctgtacaaccgaccgcttaccgaaggtgagtccaagattctctatgccaacgaaggtgtaatcaagccggtcggtattgttaaaggcgtttcactcgctaccaagactccggcgtcgatgcgcgttgacacagctaataaacgaggatacacaaattttccactgaaattaacgataatccacagcaatggggaggcaccgatcaggcagctgagtcggttagttatcattgtattgttgatatcggcgctattctag

SEQ ID NO: 14 - DNA, 2319 bp, Trypanosoma congolense
atgatgggatattctaaatcggttcggcagacgttgatatgcctcttgttggtcgcgattgacacgtatcactgcacaacggcgtatggatcgggaatcaggggggaggaggagaagaatcgaagcttgttcctcccaggggggctttggtacaagaaagatgagtggaaggatggcaattggctgcaatctaaagagtggaaagccgggtatgcgtggtggccgtggcggtcgtggtgctccaataaaactatcggtgcgtccgggaaagaactttgccgcaaggaatgggactctcagagggaaaaaggatacacactcaagccaagggaaagcgtgctctttagggagagcagtggcacaaagagaatgcgccgcgttcactcctttcgcataccttcgatggttgaggcgaatggtgtgttaattggcattgcagatgcgcgatatttgagctccgcggacttcactttcatcgacacggttgccaagtacagtgctgacggcggtgagacgtggaaaactgaagtcatcattgaaaatgctcgtgtggattcgtttcactcccgcgtggtggatcccacagttgccgtgaagaataacagtatttatgtgcttgttggaaggtacaacatctccaaagcgtattggacgtggcaacactacggcaacgattgggacatacttatgtacaagggtaccgtaaccaagacctccgaagatggcaaacctgccgcaaacattgagtggacctcggtattaaaccttaagtcgcttctggaaaccggactatacgttggagggcatgaggctacgcagtttcttggtggtgtaggtaatactgtcgtaacaccggacggcacgatcgtatttcccatccaggtaaaaaattcatggaatcatgtcgctgcaatgataatgtattcgagtgacgatggcgccacgtggcacttgggaggaggcgcaacacccgttggtacaacagaggcatccgctatctggtgggacggcaaactggttctcaattgcagaacggacttggggtaccgcaaagtattcgagacaaccgaccttgggacaacgtggaaagagtcattaggggcgctctctcgtgtgattggtaactcgccagaccgcaagcagcctggaagctcaggcagtgctattactttggaggtcgagggtgtgcaagtcatgcttattacgcagcctaagaatacaaaaacgaggtatagtcgtgatcgcctacaactgtggctgagtgatggcagtcgtgtgtggttagtcggacagatatcacgaggcgatgacaacagtccctacagctcgctgctgtacactagtgacgataagctttattgcctgtatgaacaaaatattgaagaggtgtacagtatttaccttgtccatctcgttgacgaactggagaaaatcaaagcgaccgtgagattatggaaggagcaggatgcactcctctctggcaattgctctgcaactgccgaggatggcagcgattgcaacggcgtgcccaccgctggcctcgtcgggctgctttccggacctgcgcaaggaaacgcgtggcctgatgcatataattgtgtaaatgccagcctcgtgaatgttacaagtgatgcagatggtctgcagcttagcgggttgaacaggggccgtgtgtcgtggccggtgcgtgcacagggccaggatcagcggtattactttgcgaccgttcggttcactctggtcgcaactgtgcagttaatgaaggcaccaaatagcaacgttgccgtgttggggtttggaaattcaaaaggagaaaatctgaccttgtgggtggcaagtacaacgtggaccctgacgtacggaggcgagagaaaaaaagttgttgcgccgtcactgagcagcgatgcatccgttcaatttgcgctgatacttaatggtggcagtgtgtccgtgtacgctgatggcgtacatgtaccgcaactcgataaaagggttgctgccaaaaataaattgttaaatattgatcatttcttcgccgaaagcaactatatgggagatacaaacaacatattcacaaagaacatgctcctgtataaccggaaactgagtgaaagcgagctcaaactgttgtccctcaaccgggaggctataagagctgctgacgggttaaattacttaaaagagcagcaaggtggctccgagagcgaaataaagtctacttcggattccaacgtcagcgatcctgctgacaacgaaacctctgaaaagatgtttctgcaagtggctctgattttgcttctggttataggacagaactga

SEQ ID NO: 15 - protein, 734 aa, Trypanosoma congolense
MWPVNCYALLALVVAGQCCGPMHATAAVGTTHQALLWGSKWALRNKTTPKDGEVWWSNPQPGWKEVYDDEWEEWFMEQEGPTGVDGVRAEWYRRMKDGYILVGGPKLNSPDMNSTGMTMRTVHSYRIPSIVEVGGVLMCVGDARYITSTDYFFTDTVAAYSTDGGRTWKREVIIPNGRVDAHYSRVVDPTVVAKGNNIYVLVGRYNVTRGYWHNQNDEAAIADWEPFVYKGTVNVGTKDNATDVSISWERTALKSLYNFPVSGSPGTQFLGGAGGGVVTSNGTIVLPVQARNKANRVVSMILYSADDGKSWHFGKGEAGVGTSEAALTEWDGKLLISARSDGGQGYRMIFESSDLGATWKEMLNSISRVIGNSPGRSGPGSSSGFITVTVEGVPVMLLTHPKNLKGVYSRDRLQMWMTDGNRMWHVGQVSEGDDNSAYSSLLYTPDGVLYCLHEQNIDEVYSLHLVRLVDELKSIKSTALVWKAQDELLLGNCLPGDKYDPGCDGIPTAGLAGLLVGPLTEKTWPDAYRCVNAATSGAVSTAEGVRLDVGGGGHVVWPVSEQGQDQRYYFTNSEFTLAVTVRFDEMPRGELPLLGFVNRKGKVNKILKVSLSGVEWLLAYGNEYNSTAAEPLSVNESHQVVLTLHDGIVSLHVDGGNTTATVSVRVASPEELLNIHHLFVGTPVDGGAKEHVNITVSNVLVYNRPLRGVELLGLFANRGRIRVPGSDNGVLSGG

SEQ ID NO: 16 - protein, 734 aa, Trypanosoma congolense
MWPVNCYALLALVVAGQCCGPMHATAAVGTTHQALLWGSKWALRNKTTPKDGEVWWSNPQPGWKEVYDDEWEEWFMEQEGPTGVNGVRGEWYRRMTDGYILVGGPKLNSPDMNSTGTTMRTVHSYRIPSIVEVGGVLMCVGDARYITSTDYFFTDTVAAYSTDGGRTWKREVIIPNGRVDAHYSRVVDPTVVAKGNNIYVLVGRYNVTRGYWHNRNDKAAIADWEPFVYKGTVNVGTKGTATDVSISWERTALKSLYNFPVSGSPGTQFLGGAGGGVVTSNGTIVLPVQARNKANRVVSMILYSADDGKSWHFGKGEAGVGTSEAALTEWDGKLLISTRSDGGQGYRMIFESSDLGATWKEMLNSISRVIGNSPGRSGPGSSSGFITVTVEGVPVMLLTHPKNLKGVYSRDRLQMWMTDGNRMWHVGQVSEGDDNSAYSSLLYTPDGVLYCLHEQNIDEVYSLHLVRLVDELKSIKSTALVWKAQDELLLGNCLPGDKYDPGCDGIPTAGLAGLLVGPLTEKTWPDAYRCVNAATSGAVSTAEGVRLDVGGGGHVVWPVSEQGQDQRYYFTNSEFTLAVTVRFDEMPRGELPLLGFVNRKGKVKKILKVSLSGVEWLLAYGNEYNSTAAEPLNVNESHQVVLTLHDGIVSLHVDGGNMTATVSVRVASPAELLNIHHLFVGTPVDGGAKEHANITVSNVLVYNRPLRGVELLGLFANRGRIRVPGSDNSVLSGG

SEQ ID NO: 17 - protein, 734 aa, Trypanosoma congolense
MRPVNCYALLALVVAGQCCGHMHATAAVGTTHQALLWGSKWALRNKTTPKDGEVWWSNPQPGWKEVYDDEWEEWFMEQEGPTGVNGVRGEWYRRMKDGYILVGGPKLNSPDMNSTGTTMRTVHSYRIPSIVEVGGVLMCVGDARYITSTDYFFTDTVAAYSTDGGKTWKTEVIIPNGRVDAHYSRVVDPTVVAKGNNIYVLVGRYNVTRGYWHNQNDEAAIADWEPFVYKGTVNVGTKGTATDVSISWERTALKSLYNFPVSGSPGTQFLGGAGGGVVTSNGTIVLPVQARNKANRVVSMILYSADDGKSWHFGEGEAGVGTSEAALTEWDGKLLISTRSDGGQGYRMIFESSDLGATWKEMLNSISRVIGNSPKRNGPGSSSGFITVTVEGVPVMLLTHPKNLKGSYYRDRLQMWMTDGNRMWHVGQVSEGDDNSAYSSLLYTPDGVLYCLHEQNIDEVYSLHLVRLVDELKSIKSTALVWKAQDELLLGNCLPGDKYDPGCDGIPTAGLAGLLVGPLTEKTWPDAYRCVNAATSGAVSTAEGVRLDVGGGGHVVWPVSEQGQDQRYYFTNSEFTLAVTVRFDEMPRGELPLLGFVNHKGKVNKILKVSLSGVEWLLAYGNEYNSTAAEPLSVNESHQVVLTLHDGIVSLHVDGGNTTATVSVRVASPEELLNIHHLFVGTPVDGGAKEHANITVSNVLVYNRPLRGVELLGLFANRGRIRVPGSDNGVLSGG

SEQ ID NO: 18 - protein, 778 aa, Trypanosoma congolense
MSHCFVPAWSKALCLLLLSHCLYLAHASGNGRTTRELFLGGGHWVVGKECLAVNEEGSAARTLECNGNCSPDEDSQRRSADDNDGLQEETINCVLEPRSKQLGVAKDMEGKHVVDSFRIPSIVEVDGVLITVSDVRYLNSNDLSFIDTVARYSADGGRTWETEVIIKNARVNAEHSRVVDPTVVVKNNTIFVLVGRYNKSDAYWTWQGGGGDWDILMHKGTVTKSLRGGKPSVNIEWDEPQNLKYLLSTVGKIDGRSLIQYIGGVGNCVVTPNGTIVLPVQVLNTNRSVMAMIIYSTDEGESWQFSKSATPVGTTESSIVWWDDKLLLNGRTNNDLGYRKVYESSDLGTTWTEVVGTISRVIGNSPGRNQPGSSGSSIAITLEGMRVMLITQPKNIKGSWHRDRLQLWLTDGNRVWLVGQISEGDDNGPYSSLLYTSNGTLYCLYEQDKAAVLSIYLIKLEDELESIKSIVKLWKDQDALLSGNCSSPDGDYTEGCVGIPTAGLVGLLSGPSDEDVWHDAYRCVDASVENVVNVADGLQLSGWNSSRVLWPVSSQGQDQKYHFADVHFTLVANLRLVGAPKGDFSLVGFEMYEGETRKTVKLSAIKSALWEMCHTDLTTRGSRGSLPCDEVHQVALTLRNGVISVYANGRHLSVLDTKVAGANELLNISNFFVGHPGVGGALPWGSAVVRDVLLYNRPLHETELESLYLNGDVIKVVNHGAAGISAARDAELLHVRGDGGDKPDAVPLKLAIITGDGVVRFRGLYQMASLVLLGLMLS

SEQ ID NO: 19 - protein, 778 aa, Trypanosoma congolense
MSHCFVPVWSKALCLLLLSHCLYLAHASGNGRTTRELFLGGGHWVVGKECLAVNKEGSVAQTLECNGNCSPDEDSRIRSADDNDGLQEETINCALEPRSEQLGVAKDMEGKHIVDSFRIPSIVEVDGVLITVSDVRYLNSNDLSFIDTVARYSADGGRTWETEVIIKNARVNAEHSRVVDPTVVVKNNTIFVLVGRYNKSDAYWTWQGGGGDWDILMHKGTVTKSLRGGKPSVNIEWDEPQNLKYLLSTVGKIDGRSLIQYIGGVGNCVVTPNGTIVLPVQVLNTNRSVMAMIIYSTDEGESWQFSKSATPVGTTESSIVWWDDKLLLNGRTNNDLGYRKVYESSDLGTTWKEVVGTISRVIGNSPGRNQPGSSGSSIAITLEGMRVMLITQPKNIKGSWHRDRLQLWLTDGNRVWLVGQISEGDDDGPYSSLLYTSNGTLYCLYEQDKSAVLSIYLIKLEDELESIKSIVKLWKDQDALLSGNCSSPDGDYTEGCVGIPTAGLVGLLSGPSDGDVWHDAYRCVDASVDNVVNVADGLQLSGWNSSRVLWPVSSQGQDQKYHFANVHFTLVANLRLVGAPKGDFSLVGFETYEGERRKTVKLSAIKSAFWEMCHTDLTTRGSRGSPPCDEVHQVALTLRDGVISVYANGRHLSVLDTKIAGANELLNITNFFVGHPGVGDALPWGSAVVRDVLLYNRPLHETELESLYLNGDVIKVVNHGAAGISAARDAELLHVREDGGDKPNAVPLKLAIITDDGVARFRGLYQMASLMLLGLMLS

SEQ ID NO: 20 - protein, 738 aa, Trypanosoma congolense
MPSYLLPAAVVLYCFGGLVPAAQCISTTREAFMEAGQWSVNKDCLITAEGSRRSKASGSYEKGYASVESTTKDVNTRGGVQTSEACTLEPEVRDNSTSGDGKERHLIHSFRIPSLVEIDGVLIATFDTRYLHASDSSLIDTAMKYSADQGKTWKTEIIIKNARLTDNFSRVVDPTVVVKGDNLFIFVGRYNTSSTPWVWQKNGKDWDVLLYKAKVRKESAGGVPSVSFTWDEPLHLKHLLTSVGKIDGRSLIQYIGGVGNGIVTPKGTIVFPVQVLNTNKSVMNMLLYSSNDGKTWEFSKTSTPAGTTEASLVWWDGQLLLTSRTTPDVGSRKVYLTSDLGTSWNEAIGSISRVIGNSRYRNDPGGSGSSIAITVEGVPVMLVTHPENAKGRWNRDRMRLWLTDGNRMWLVGQISEGDDNSAYSYLLYTKNGTLLCLYERNIREIYSIYLARLEDEMEDIKSTVRLWKAHDELLSGDCQLNKKRRSGCTGIPITGLVGLLAGLPRKSVWPDAYNCVDASISKNNKQVSHDPPSRSTMKRRVVWPVGDQGQDQRYHFVNTHFTFVATIYFDRAPQEVSLMGFENNEESTKTLTVSIGNGRLVLTYGGLLEEIPMTRLDWSVTHQVALTLHNGEVSLHVDGNPSIANVRLKLHEPDRLLNISNLFTSTPAPVKTGKGSTVTVNNVILYNRMLNETELARLFNSRDLIDEVGDVHPVSGGGVGEWRFHVWILLAAYVLVAY

SEQ ID NO: 21 - protein, 787 aa, Trypanosoma congolense
MGYSKSVRQTLICLLLVAIDTYHCTTAYGSGIRGEEEKNRSLFLPGGLWYKKDEWKDGNWLQSKEWKAGYAWWPWRSWCSNKTIGASGKELCRKEWDSQREKGYTLKPRESVLFRESSGTKRMRRVHSFRIPSMVEANGVLIGIADARYLSSADFTFIDTVAKYSADGGETWKTEVIIENARVDSFHSRVVDPTVAVKNNSIYVLVGRYNTSNTYWTMQNNGNDWDILMYKGTVTKTSEDGKPAANIEWTGTQNLKYLLKLVDQIEGKSLTQFLGGVGNTVVTPDGTIVFPIQVKNSWNQVAAMIMYSSDDGATWHLGGGATPVGTTEASAIWWDGKLVLNCRTDLGYRKVFETTDLGTTWKESLGALSRVIGNSPDRKQKGSSGSAITLEVEGVQVMLITQPKNTKGDYNRDRLQLWLSDGSRVWLVGQISRGDDNSPYSSLLYTSDDKLYCLYEQDIRGVLSIYLVHLVDELEKIKATVRLWKEQDALLSGNCSATAEDGSDCNGVPTAGLVGLLSGPAQGNAWPDAYNCVNASLVNVTSDADGLQLGGLNRGRVSWPVRAQGQDQRYYFANVRFTLVATVRLNGISNLEIPLMGFENFQKSTRDTLIVSIVDDAYWSKCKEGPVPGVNVDAPECQKFHQVAIMFQNGRVSVYADGIHIPQLDTSIVDASALLNISSFFLGHPEVGSRFTSADVIVKNVLLYNRPLTEGESKILYANEGVIKPVGIVKGVSLATKTPASMRVDTANKRGYTNFPLKLTIIHSNGEAPIRQLSRLVIIVLLISALF

SEQ ID NO: 22 - protein, 771 aa, Trypanosoma congolense; identical to SEQ ID NO: 4

SEQ ID NO: 23 - protein, 20 aa, Trypanosoma vivax
RTSIDYHLIDTVAKYSADDG

SEQ ID NO: 24 - protein, 19 aa, Trypanosoma congolense
PKNIKGSWHRDRLQLWLTD

SEQ ID NO: 25 - protein, 19 aa, Trypanosoma vivax
PVSAQGQDHRYEAANAEHT

SEQ ID NO: 26 - protein, 12 aa, Trypanosoma congolense
KVDGTALSSARN

SEQ ID NO: 27 - protein, 16 aa, Trypanosoma congolense
KIESTGAVEGGGVVKN

SEQ ID NO: 28 - protein, 12 aa, Trypanosoma congolense
KVTWGTQVPLKG

SEQ ID NO: 29 - protein, 13 aa, Trypanosoma congolense
REWVEAVTSYSRV

SEQ ID NO: 30 - protein, 13 aa, Trypanosoma congolense
KVGDVLVAIGERR

SEQ ID NO: 31 - protein, 14 aa, Trypanosoma congolense
KLYNFVTATVEGRR

SEQ ID NO: 32 - protein, 15 aa, Trypanosoma congolense
RISYFYIGGYGNPRT

SEQ ID NO: 33 - protein, 12 aa, Trypanosoma congolense
RGWDPLLIVGKV

SEQ ID NO: 34 - protein, 12 aa, Trypanosoma congolense
KILEVYNADGRW

SEQ ID NO: 35 - protein, 19 aa, Trypanosoma congolense
KENIPVLAVAVNEGDSNKI

SEQ ID NO: 36 - protein, 30 aa, Trypanosoma congolense
KIGYLRDEFSSSVPYDIGNIAQLDDALAKI

SEQ ID NO: 37 - protein, 16 aa, Trypanosoma congolense
KDGEVWWSNPQPGWKE

SEQ ID NO: 38 - protein, 12 aa, Trypanosoma congolense
KGNNIYVLVGRY

SEQ ID NO: 39 - protein, 15 aa, Trypanosoma congolense
RVVSMILYSADDGKS

SEQ ID NO: 40 - protein, 15 aa, Trypanosoma congolense
RMIFESSDLGATWKE

SEQ ID NO: 41 - protein, 12 aa, Trypanosoma congolense
RLQMWMTDGNRM

SEQ ID NO: 42 - protein, 23 aa, Trypanosoma congolense
RLDVGGGGHVVWPVSEQGQDQRY

SEQ ID NO: 43 - protein, 13 aa, Trypanosoma congolense
RGVELLGLFANRG

SEQ ID NO: 44 - protein, 14 aa, Trypanosoma congolense
KGTATDVSISWERT

SEQ ID NO: 45 - protein, 14 aa, Trypanosoma congolense
RMKDGYILVGGPKL

SEQ ID NO: 46 - protein, 26 aa, Trypanosoma congolense
RVASPEELLNIHHLFVGTPVDGGAKE

SEQ ID NO: 47 - protein, 24 aa, Trypanosoma congolense
RYITSTDYFFTDTVAAYSTDGGRT

SEQ ID NO: 48 - protein, 10 aa, Trypanosoma congolense
RVIGNSPGRS

SEQ ID NO: 49 - protein, 28 aa, Trypanosoma congolense
RSGPGSSSGFITVTVEGVPVMLLTHPKN

SEQ ID NO: 50 - protein, 13 aa, Trypanosoma congolense
RGELPLLGFVNRK

SEQ ID NO: 51 - protein, 28 aa, Trypanosoma congolense
RNGPGSSSGFITVTVEGVPVMLLTHPKN

SEQ ID NO: 52 - protein, 14 aa, Trypanosoma congolense
RGELPLLGFVNHKG

SEQ ID NO: 53 - protein, 14 aa, Trypanosoma congolense
RMTDGYILVGGPKL

SEQ ID NO: 54 - protein, 14 aa, Trypanosoma congolense
KAAIADWEPFVYKG

SEQ ID NO: 55 - protein, 26 aa, Trypanosoma congolense
RVASPAELLNIHHLFVGTPVDGGAKE

SEQ ID NO: 56 - protein, 15 aa, Trypanosoma congolense
RIRVPGSDNSVLSGG

SEQ ID NO: 57 - protein, 26 aa, Trypanosoma congolense
KEVYDDEWEEWFMEQEGPTGVDGVRA

SEQ ID NO: 58 - protein, 20 aa, Trypanosoma congolense
KGEAGVGTSEAALTEWDGKL

SEQ ID NO: 59 - protein, 17 aa, Trypanosoma congolense
KAPNSNVAVLGFGNSKG

SEQ ID NO: 60 - protein, 14 aa, Trypanosoma congolense
KVFETTDLGTTWKE

(19) United States
(12) Patent Application Publication
(10) Pub. No.: US 2012/0276131 A1
(43) Pub. Date: Nov. 1, 2012

(54) ANTI-TRYPANOSOMIASIS VACCINES AND DIAGNOSTICS

(76) Inventors: Virginie Coustou Linares, Marcheprime (FR); Theo Baltz, Bordeaux (FR); Nicolas Plazolles, Villenave d'Ornon (FR)

(21) Appl. No.: 13/508,851

(22) PCT Filed: Nov. 10, 2010
(86) PCT No.: PCT/EP10/67245

§ 371 Date: Jul. 12, 2012

(30) Foreign Application Priority Data
Nov. 10, 2009 (FR) 0957953

(51) Int. Cl. C12N 9/24; A61K 39/005; C07K 16/20; C12N 15/11; A61P 37/04; G01N 33/573; C12N 15/63; C12N 5/10; C12N 1/15; C12N 1/19; C12N 1/21; C12N 15/52; A61P 33/00

(52) U.S. Cl. 424/191.1; 536/23.2; 530/387.9; 536/24.31; 435/200; 435/320.1; 435/325; 435/348; 435/254.11; 435/254.2; 435/252.33; 435/252.3; 435/7.4

(57) ABSTRACT

The present invention has as an object a novel genetic material coding for trans-sialidase-like proteins of African trypanosomes, and relates to the use of said genes and proteins in vaccines, therapeutics and diagnostics. The present invention also relates to the immunization of human and/or nonhuman animals against trypanosomosis.

The present invention relates to a novel genetic material coding for trans-sialidase-like proteins of African trypanosome parasites, and concerns the use of said genes and proteins in vaccines, therapeutics and diagnostics. The present invention also relates to the immunization of humans and/or non human animals against trypanosomosis and trypanosomiasis.

Trypanosomosis and trypanosomiasis are caused by several species of parasitic protozoa of the genus Trypanosoma, and African trypanosomes generally refer to trypanosomes belonging to the group Salivaria, which itself includes three principal sub-genera: Trypanozoon, Duttonella and Nannomonas.

Only the sub-genus Trypanozoon comprises, in addition to species infectious to animals, two species infectious to humans in whom they cause sleeping sickness. The other sub-genera include species that infect wild and domestic animals and are never infectious to humans, but which can have significant indirect health consequences.

The sub-genus Trypanozoon consists of polymorphic trypanosomes (long and short or stumpy forms), with an optional free flagellum and a small kinetoplast in the subterminal (posterior) position. The species of this sub-genus are Trypanosoma (T.) brucei, T. evansi and T. equiperdum. T. brucei includes three subspecies: T. b. brucei, T. b. gambiense and T. b. rhodesiense, which are quite similar in morphological, antigenic and biochemical terms and are distinguished by their infectious nature, their pathogenicity and their geographical distribution. T. brucei and its subspecies are transmitted by tsetse flies. T. evansi is transmitted to cattle, horses and camels by biting flies other than tsetse (Tabanidae) in Africa, South America and Southeast Asia. T. equiperdum has no invertebrate host (sexual transmission in horses). The latter two species extend far beyond areas with tsetse flies and are cosmopolitan. Their morphology is similar to that of T. brucei but they are monomorphic (long forms only).

Trypanosomes belonging to the sub-genus Duttonella are club shaped, with a round and broad posterior extremity and a body that narrows toward the anterior extremity. The kinetoplast is voluminous, round and in the terminal position; the undulating membrane is relatively undeveloped, narrow and terminates in a free flagellum. T. vivax and T. uniforme are species of parasites of wild and domestic ruminants. They can be transmitted mechanically or by tsetse flies, in which they colonize exclusively the proboscis and proventriculus.

Trypanosomes of the sub-genus Nannomonas are small (8-24 μm), and they have no free flagellum at any stage of their development. The average-size kinetoplast is in the subterminal or marginal position. The posterior extremity is round and the undulating membrane narrow. Their pathogenicity in Africa is significant for cattle, pigs and dogs. Their development in the tsetse fly takes place exclusively in the stomach and proboscis. The principal species are T. congolense and T. simiae. These trypanosomes are small with a round posterior extremity, a kinetoplast in the marginal position and a narrow undulating membrane.

Domestic ruminants in Africa are primarily infected by three species of pathogenic trypanosomes, T. congolense, T. vivax and T. brucei, which are responsible for a pathology called nagana. Other animals are infected by another pathogenic trypanosome species, T. evansi, which is responsible for a pathology called surra. Trypanosomes are characterized by a large genetic diversity, which relates to their infectivity, virulence, pathogenicity, transmissibility and sensitivity to trypanocidal products.

T. congolense is the principal agent of bovine trypanosomosis in Africa, by its frequency and pathogenicity. It also adapts to various nonhuman animal species, and can thus indifferently parasitize bovids, suids, ovids, caprids, equids and canids.

T. brucei, and notably the subspecies Trypanosoma brucei gambiense, is probably the most widely known since it is responsible for the chronic form of sleeping sickness in man in Western and Central Africa. The subspecies Trypanosoma brucei brucei is a parasite of domestic and wild animals throughout Africa, but it is not infectious to man due to the lytic effect of apolipoprotein L, present in human serum, on the blood forms of these trypanosomes. The third subspecies is Trypanosoma brucei rhodesiense, which is the agent of sleeping sickness in its acute form in Africa.

Additionally, the subspecies T. evansi is transmitted to bovids, horses and camels and has significant economic repercussions in Africa, notably for the breeding of cattle and buffaloes.

Lastly, T. vivax is a parasite primarily of ungulates in tropical Africa and is transmitted by horseflies and gadflies.

Trypanosomes have a complex life cycle which includes various morphological forms. They have in general a fusiform body and a flagellum connected to the body by an undulating membrane. They reproduce asexually by binary fission. During an infection, the tsetse fly (Glossina sp.) injects into the dermis of the host at the puncture site the infectious metacyclics present in the mouthparts. The parasites multiply in the dermis at the inoculation point. A local reaction related to parasite multiplication in the dermis occurs, and the parasites give rise to blood forms. This stage can last from 1-3 weeks, for example, in the case of T. congolense. Then, the parasites invade the blood, the lymphatic system, in particular the lymph nodes, and various organs such as the liver, spleen, heart, kidneys and testicles, which then exhibit significant lesions. The tsetse becomes infected by and feeds on parasitized animals. Once infected, it remains infectious throughout its life. In the case of T. brucei and T. congolense, the trypanosome undergoes in the insect a complex cycle involving dedifferentiation in the intestine into noninfectious procyclic forms. In the salivary glands or mouthparts, trypanosomes transform into adherent epimastigote forms which actively multiply. Their differentiation leads to the infectious stage represented by metacyclic forms, which divide no further.

The T. vivax cycle comprises no procyclic stage. It begins with flagellum attachment in the blood forms introduced by the tsetse. They differentiate into epimastigote forms, which proliferate and then differentiate into infectious metacyclics. The total duration of the cycle in the tsetse is roughly 5-10 days for T. vivax, 18 days for T. congolense and 30 days for T. brucei.

The sources of infection for domestic animals are other domestic animals or wild animals that are sick or are healthy carriers. The existence of the reservoir comes from the fact that certain species are relatively unreceptive to the infection, and relatively insensitive to the disease.

Potential vectors vary by trypanosome species. T. congolense and T. brucei are transmitted exclusively by biological vectors such as tsetse flies, but T. vivax can also be transmitted by mechanical vectors such as biting flies (gadflies or stable flies). T. evansi is transmitted exclusively by mechanical vectors. Transmission efficiency depends on tsetse infection rates and host-vector interactions. Generally, trypanosomes that are infectious to animals have higher infection rates than trypanosomes that infect man, which contributes to the very wide distribution of animal trypanosomosis.

Analysis of trypanosomes by electron microscopy shows the existence of a roughly 15 nm coat covering the totality of the cell body of the parasite. This coat is present only on the surface of the blood and metacyclic forms. It is comprised essentially of a variable surface glycoprotein (VSG) with other membrane proteins in small quantities. VSGs form a very dense structure comprising a physical barrier between the plasma membrane and the host. The 3-D structure predicts that only a small part of the protein is exposed on the surface of the parasite. Thus, the principal role of the coat is to mask the invariant membrane antigens of the parasite by presenting several immunodominant motifs to the immune defenses of the host. The coat further protects blood forms against lysis by activation of the alternate complement pathway.

The fight against animal trypanosomosis depends on the screening of animals and treatment on the basis of cost recovery. The principal chemical compounds used to treat trypanosomosis are diminazene aceturate, homidium bromide or chloride, isometamidium chloride, quinapyramine, suramin and melarsomine. However, no new molecule has been placed on the market for at least 30 years, whereas the past few years have seen a fresh outbreak of the disease due to the appearance of trypanocide resistances and the extensive and occasionally inappropriate use of drugs leading to the selection and amplification of resistances reported notably in all regions of Africa affected by the disease.

### SUMMARY OF THE INVENTION

The Applicant identified and obtained a novel genetic material coding for novel trans-sialidase-like proteins named TcoTS-like 1, 2, and 3, recognized by anti-African trypanosome antisera. The genetic material can be used to produce proteins and polypeptides intended for the development of diagnostic tests and for the preparation of vaccine or pharmaceutical compositions against infections by African trypanosomes. Similarly, the protein and any corresponding polypeptide fragment can be used for the production of specific antibodies against the parasite, for the purpose of diagnostics or passive immunization.

### BRIEF DESCRIPTION OF THE FIGURES

FIG. 1: represents the nucleotide sequence coding for the trans-sialidase-like protein TcoTS-like 1;

FIG. 2: represents the nucleotide sequence coding for the trans-sialidase-like protein TcoTS-like 2;

FIG. 3: represents the nucleotide sequence coding for the trans-sialidase-like protein TcoTS-like 3;

FIG. 4: represents the peptide sequence corresponding to the trans-sialidase-like protein TcoTS-like 1;

FIG. 5: represents the peptide sequence corresponding to the trans-sialidase-like protein TcoTS-like 2;

FIG. 6: represents the peptide sequence corresponding to the trans-sialidase-like protein TcoTS-like 3;

FIG. 7: represents a sequence alignment between the trans-sialidase-like protein TcoTS-like 2 and a trans-sialidase protein of the parasite Trypanosoma cruzi (T. cruzi TS);

FIGS. 8A and 8B: represent a diagram of the five subfamilies of trans-sialidase-related proteins of the parasite T. congolense; the percent identities between genes of the same subfamily are indicated (FIG. 8A) with a table showing the percent identities between said proteins (FIG. 8B);

FIG. 9: represents the nucleotide sequence coding for the TcoTS-A1 protein;

FIG. 10: represents the nucleotide sequence coding for the TcoTS-A2 protein;

FIG. 11: represents the nucleotide sequence coding for the TcoTS-A3 protein;

FIG. 12: represents the nucleotide sequence coding for the TcoTS-B1 protein;

FIG. 13: represents the nucleotide sequence coding for the TcoTS-B2 protein;

FIG. 14: represents the nucleotide sequence coding for the TcoTS-C protein;

FIG. 15: represents the nucleotide sequence coding for the TcoTS-D1 protein;

FIG. 16: represents the nucleotide sequence coding for the TcoTS-D2 protein;

FIG. 17: represents the peptide sequence corresponding to the TcoTS-A1 protein;

FIG. 18: represents the peptide sequence corresponding to the TcoTS-A2 protein;

FIG. 19: represents the peptide sequence corresponding to the TcoTS-A3 protein;

FIG. 20: represents the peptide sequence corresponding to the TcoTS-B1 protein;

FIG. 21: represents the peptide sequence corresponding to the TcoTS-B2 protein;

FIG. 22: represents the peptide sequence corresponding to the TcoTS-C protein;

FIG. 23: represents the peptide sequence corresponding to the TcoTS-D1 protein;

FIG. 24: represents the peptide sequence corresponding to the TcoTS-D2 protein;

FIGS. 25A and 25B: represent a sequence alignment between 11 trans-sialidase-related proteins of the parasite Trypanosoma congolense;

FIG. 26: represents a table showing the percent identities between trans-sialidase-related proteins of the parasites T. congolense and T. brucei.

FIG. 27: represents a table of the various peptides identified in the immunoprecipitation experiment with anti-TcoTS-A1 serum; their relation to proteins TcoTS-A1, TcoTS-A2 or TcoTS-A3 (A), TcoTS-like 2 (B) and TcoTS-D2 (C).

FIG. 28: represents a table of the various peptides identified in the experiment involving T. congolense blood form membrane preparations (A), their relation to TcoTS-A1, TcoTS-A2 or TcoTS-A3 proteins is illustrated by a plus sign (+); and a table of peptides related to the TcoTS-like 2 protein identified during immunoprecipitation experiments with anti-peptide 1, anti-peptide 2 or anti-peptide 3 sera (B).

FIGS. 29A and 29B: represent measurements of hematocrit (A) and mean survival (B) in mice after immunization with TcoTS-like 2, TcoTS-A1 and TcoTS-B1 proteins or with BSA. The number of mice (n) used during the various immunizations is indicated.

### DEFINITIONS

“African trypanosomes” refer to parasitic protozoa of the genus Trypanosoma belonging to the group Salivaria, which itself includes three principal sub-genera: Trypanozoon, Duttonella and Nannomonas, such as defined above. These have been described as African trypanosomes, but however are found today in Asia and South America as well as on the African continent. The most common African trypanosomes are Trypanosoma congolense, Trypanosoma vivax, Trypanosoma evansi and Trypanosoma brucei.

The terms “trypanosomosis” and “African animal trypanosomosis” (AAT) generally refer to infections of nonhuman animals caused by African trypanosomes, whereas the terms “trypanosomiasis” or “African trypanosomiasis” are used to refer to human infections also caused by African trypanosomes. For purposes of simplification, the terms trypanosomosis and trypanosomiasis are used indifferently herein.

### DETAILED DESCRIPTION OF THE INVENTION

The present invention has as an object a DNA or RNA molecule coding for novel trans-sialidase-like proteins called TcoTS-like 1, 2, and 3, and belonging to African trypanosomes. These novel DNA or RNA molecules comprise at least one strand comprising a nucleotide sequence selected from the sequences SEQ ID NOs: 1-3, a sequence complementary, antisense or equivalent to one of the sequences SEQ ID NOs: 1-3, and notably a sequence comprising an identity of at least 70% with one of the sequences SEQ ID NOs: 1-3, or a sequence having, on a sequence of 100 contiguous nucleotides, at least 50%, preferably at least 60%, or at least 70%, or at least 80%, 85%, 90%, 91%, 92%, 93%, 94%, or 95% homology with said sequences, or a nucleotide sequence able to hybridize with one of the sequences SEQ ID NOs: 1-3 under stringent hybridization conditions.

Stringent hybridization conditions refer to hybridization at a temperature of 65° C. overnight in a solution containing 0.1% SDS, 0.7% dried skimmed milk and 6×SSC, followed by washings at room temperature in 2×SSC—0.1% SDS and at 65° C. in 0.2×SSC—0.1% SDS.

The invention also relates to DNA or RNA fragments whose nucleotide sequence is identical, complementary, antisense or equivalent to any one of the following sequences: SEQ ID NO: 1, SEQ ID NO: 2 or SEQ ID NO: 3, and notably DNA or RNA fragments, for any sequence of 30 contiguous monomers, at least 50%, preferably at least 60%, or at least 85%, 90%, 91%, 92%, 93%, 94%, or 95% homology with any one of said sequences.

Nucleotide sequence refers to at least one strand of DNA or its complementary strand, or one strand of RNA or its antisense strand or their corresponding complementary DNA. The DNA sequence as represented in one of the sequences SEQ ID NOs: 1-3 corresponds to the messenger RNA sequence, given that that the thymine (T) in DNA is replaced by uracil (U) in RNA.

According to the invention, two nucleotide sequences are said to be equivalent with respect to each other as a result of natural variability, notably spontaneous mutation of the species from which they were identified, or induced variability, as well as homologous sequences, with homology being defined below. Variability refers to any spontaneous or induced modification of a sequence, notably by substitution and/or insertion and/or deletion of nucleotides and/or nucleotide fragments, and/or extension and/or shortening of the sequence at least one end, or unnatural variability that may result from the genetic engineering techniques used. This variability can be expressed by modifications of any starting sequence, regarded as a reference, and can be expressed by a degree of homology in relation to said reference sequence.

Homology characterizes the degree of identity of two compared nucleotide (or peptide) fragments; it is measured by percent identity, which is notably determined by direct comparison of nucleotide (or peptide) sequences in relation to reference nucleotide (or peptide) sequences.

Another object of the invention relates to proteins called TcoTS-like 1, TcoTS-like 2 and TcoTS-like 3, with apparent molecular weights of roughly 85 kDa for the TcoTS-like 1 protein, roughly 76 kDa for the TcoTS-like 2 protein and roughly 78 kDa for the TcoTS-like 3 protein, and recognized by anti-African trypanosome antisera, as well as the antigenic peptide fragments thereof or an immunological equivalent of said proteins or fragments. The amino acid sequences of said proteins are represented in the sequences SEQ ID NOs: 4-6 and further comprise protein sequences that are at least 70%, 75%, 80%, 85%, 90%, or at least 95% homologous.

The proteins newly characterized by the Applicant have at the C-terminus a conserved lectin part aimed at allowing binding to sialic acids of infected animals and at the N-terminus a catalytic part with similarity to that of trans-sialidase enzymes and thus referred to as trans-sialidase-like by the Applicant.

Immunological equivalent refers to any polypeptide or peptide able to be recognized immunologically by antibodies directed against said TcoTS-like 1, 2, and 3 proteins.

The invention further relates to any fragment of TcoTS-like 1, 2 and 3 proteins, and more particularly any antigenic peptide fragment specifically recognized by anti-African trypanosome antisera.

Said proteins and protein fragments of the invention can comprise modifications, notably chemical modifications that do not alter their immunogenicity.

The present invention thus also relates to one or more peptides whose amino acid sequence corresponds to part of the sequence of the TcoTS-like 1, TcoTS-like 2 and/or TcoTS-like 3 proteins, exhibiting alone or in mixtures reactivity with the totality of sera of nonhuman animals and/or humans infected by African trypanosomes. The peptides can be obtained by chemical synthesis, by lysis of TcoTS-like 1, TcoTS-like 2 and TcoTS-like 3 proteins, or by genetic recombination techniques.

According to a second aspect, the present invention has as an object a functional expression cassette, notably in a cell from a prokaryotic or eukaryotic organism, enabling the expression of DNA coding for the totality or a fragment of the TcoTS-like 1, TcoTS-like 2 and TcoTS-like 3 proteins as described above, in particular a DNA fragment such as defined above placed under the control of the elements required for its expression. Said protein or protein fragment thus expressed is recognized by anti-African trypanosome antisera.

Generally, any cell from a prokaryotic or eukaryotic organism can be used in the context of the present invention. Such cells are known to the person skilled in the art. As examples, mention may be made of cells from a eukaryotic organism, such as mammalian cells, notably Chinese hamster ovary (CHO) cells, insect cells or fungal cells, notably unicellular or yeast cells, notably from Pichia, Saccharomyces, Schizosaccharomyces and particularly selected from the group comprised of Saccharomyces cerevisiae, Schizosaccharomyces pombe, Schizosaccharomyces malidevorans, Schizosaccharomyces sloofiae and Schizosaccharomyces octosporus. Similarly, among cells from prokaryotic organisms, mention may be made, without constituting a limitation in any way, of cells of a strain of Escherichia coli (E. coli) or enterobacteria cells. The cell can be wild-type or mutant. Mutations are described in the literature available to the person skilled in the art. Preferably, an E. coli cell is used, such as BL21 (DE3), for example.

The expression cassette of the invention is intended for the production, for example in E. coli, of TcoTS-like 1, TcoTS-like 2 and TcoTS-like 3 proteins, or fragments of said proteins, recognized by anti-African trypanosome antisera. Such antisera come from animals having contracted a recent or old infection by trypanosome species T. congolense, T. brucei, T. evansi and/or T. vivax, and contain immunoglobulins that specifically recognize TcoTS-like 1, TcoTS-like 2 and TcoTS-like 3 proteins. Also, TcoTS-like 1, TcoTS-like 2 and TcoTS-like 3 proteins can be recognized by other antibodies such as, for example, monoclonal or polyclonal antibodies obtained by immunization of varied species with the aforesaid natural protein, the recombinant protein, or the fragments or peptides thereof.

TcoTS-like 1, TcoTS-like 2 and TcoTS-like 3 proteins, or fragments thereof, refer to the antigen or antigenic fragment of natural African trypanosomes belonging to the species T. congolense, T. brucei, T. evansi and/or T. vivax, produced notably by the genetic recombination techniques described in the present application, or any fragment or mutant of said antigen on the condition that it is immunologically reactive with antibodies directed against the TcoTS-like 1, TcoTS-like 2 and TcoTS-like 3 proteins of said parasites.

Advantageously, said proteins have an amino acid sequence with a degree of homology of at least 70%, 75%, 80%, 85%, 90%, or at least 95% in relation to the sequences SEQ ID NOs: 4-6. In practice, one such equivalent can be obtained by deletion, substitution and/or addition of one or more amino acids of the native or recombinant protein. It is within the means of the person skilled in the art to carry out these modifications by known techniques without affecting immunological recognition.

In the context of the present invention, the TcoTS-like 1, TcoTS-like 2 and TcoTS-like 3 proteins can be modified in vitro, notably by deletion or addition of chemical groups such as phosphates, sugars or myristic acids, so as to improve its stability or the presentation of one or several epitopes.

The expression cassette of the invention enables the production of the TcoTS proteins TcoTS-like 1, TcoTS-like 2 and TcoTS-like 3, or an antigenic fragment of said proteins, having the amino acid sequences as specified above, and fragments of said proteins, which can advantageously be fused with an exogenous element able to contribute to its stability, purification, production or recognition. The choice of one such exogenous element is within the means of the person skilled in the art. It can be notably a hapten or an exogenous peptide.

The expression cassette of the invention comprises the elements required for the expression of said DNA fragment in the cell under study. “Elements required for the expression” refer to all of the elements that enable the transcription of the DNA fragment into messenger RNA (mRNA), such as transcription promoter sequences (CMV promoter, for example) and terminator sequences, as well as elements enabling the translation of mRNA into protein.

The present invention extends to a vector comprising an expression cassette of the invention. It can also be a plasmid vector capable of autonomous replication and in particular multiplication. It can be a viral vector and notably a baculovirus-derived vector, more particularly intended for expression in insect cells, or an adenovirus-derived vector for expression in mammalian cells.

The present invention also relates to a cell from a prokaryotic or eukaryotic organism, comprising an expression cassette, either integrated in the cell genome or inserted in a vector.

A further object of the present invention is a method for preparing one or more proteins selected from TcoTS-like 1, TcoTS-like 2 and TcoTS-like 3, or antigenic fragments of said proteins, wherein: (i) a cell from a prokaryotic or eukaryotic organism, comprising the expression cassette of the invention, is cultivated under suitable conditions; and (ii) the protein expressed by said organism is recovered.

According to a third aspect, the invention relates to monoclonal or polyclonal antibodies obtained by immunological reaction of a non human animal organism with an immunogenic agent comprised of one or more natural or recombinant TcoTS-like 1, TcoTS-like 2 and TcoTS-like 3 proteins and/or the antigenic peptide fragments thereof, such as defined above. As examples, the polyclonal antibodies of the present invention can be generated by using the TcoTS-like 1, TcoTS-like 2 and TcoTS-like 3 proteins (SEQ ID NOs: 4-6), which are injected into rabbits in order to immunize them as described in Example 2. The rabbit polyclonal sera thus obtained, designated as anti-peptide antibody 1, anti-peptide antibody 2 and anti-peptide antibody 3, respectively, are also part of the present invention given that they exhibit reactivity against their inventive peptide in indirect ELISA.

According to a fourth aspect, the present invention has as an object an active immunotherapeutic composition, notably a vaccine preparation, which comprises one or more natural or recombinant TcoTS-like 1, TcoTS-like 2 and TcoTS-like 3 proteins, and/or the antigenic peptide fragments thereof, and/or a mixture of one or more TcoTS-like 1, TcoTS-like 2 and TcoTS-like 3 proteins, and/or a mixture of one or more peptide fragments such as defined above, and optionally a suitable excipient and/or adjuvant.

The vaccine or veterinary compositions of the invention are intended to treat and/or prevent an infection by African trypanosomes in humans and/or non human animals, particularly against infections by the species T. congolense, T. brucei, T. evansi and/or T. vivax.

African trypanosomosis results in syndromes of variable gravity, ranging from acute infection with mortality in 3 to 4 weeks to chronic infection lasting months or even years. The chronic progression, characterized by intermittent parasitemias, is the most frequent in cattle. The disease begins with a hyperthermia phase, and then two to three weeks after the infecting bite the number of red blood cells and hemoglobin and hematocrit levels drop, reflecting anemia, which is the major symptom of trypanosomosis. Chronically infected animals consume less feed, become cachectic, their growth slows, and negative effects on reproduction are observed. Trypanosomosis anemia is established in two phases. During the initial phase, anemia is accompanied by parasitemia and results primarily from extra-vascular hemolysis: red blood cells are destroyed by the phagocyte system in the spleen, liver, circulating blood and bone marrow. Eventually, anemia results in bone marrow dysfunction.

Said vaccine compositions can be provided in the form of an antigenic vaccine and thus comprise a therapeutically effective quantity of one or more natural or recombinant TcoTS-like 1, TcoTS-like 2 and TcoTS-like 3 proteins, and/or the antigenic peptide fragments thereof such as described above.

The vaccine compositions can be provided in the form of DNA vaccines and can thus comprise an expression cassette, a vector, a cell from a prokaryotic or eukaryotic organism such as defined above, able to express one or more TcoTS-like 1, TcoTS-like 2 and TcoTS-like 3 proteins, and/or the antigenic peptide fragments thereof, and/or a combination thereof. The DNA vaccines can contain DNA or RNA, modified nucleotide sequences, and preferably one or more expression vectors coding for an antigenic peptide or a fragment under the control of a eukaryotic promoter sequence.

The vaccines of the present invention can be monovalent vaccines comprising a therapeutically effective quantity of one or more natural or recombinant TcoTS-like 1, TcoTS-like 2 and TcoTS-like 3 proteins, and/or the antigenic peptide fragments thereof such as described above and/or the nucleotide sequences coding for said peptide peptides or fragments.

Said monovalent vaccine prevents the infestation and thus the expression of the disease.

If said vaccine does not prevent the infestation but only the expression of the disease, it could be called an “anti-disease” vaccine. In this case, and given that differential diagnosis with other blood parasitoses is currently not systematic, the use of multivalent vaccines combining the so-called “anti-disease” vaccine with antigens of other trypanosomes and/or other therapeutic active agents and/or other vaccines commonly used in disease prevention is particularly advantageous according to the present invention.

Thus, the vaccines of the present invention can be monovalent vaccines combining one or more natural or recombinant proteins and/or peptide fragments and/or nucleotide sequence coding for said peptides and peptide fragments of one or more trypanosome species, and preferably derived from one or more similar or different trypanosome species.

Said trypanosome-derived antigenic peptides, fragments or antigenic peptide cocktails are, for example, other sialidases or trans-sialidases, tubulins, proteases, lipases and/or flagellar proteins.

As examples of trans-sialidases able to be incorporated into multivalent vaccines, mention may be made of the trans-sialidases of T. cruzi, T. congolense, T. vivax, T. evansi, T. brucei, T. rhodesiense and/or T. gambiense. Certain trans-sialidases of T. congolense, among others, are described in international application WO2004/55176 or by Tiralongo E. et al. (JBC vol. 278, No. 26, pp 23301-10, 2003). More precisely, mention may be made of T. cruzi trans-sialidase chains A and B as deposited in GenBank under numbers GI:29726491, GI:29726490, GI:29726489 and GI:29726488. It is also advantageous to use inactive mutated forms of trans-sialidases. In this respect, mention may be made of the mutant T. cruzi trans-sialidases described in international application WO2007/107488, for example, which conserve less than 20% of their sialidase and transferase enzymatic activity.

As examples of trypanosome-derived tubulins, mention may be made of T. brucei alpha-tubulin (deposited in GenBank under accession number AAA30262.1), T. brucei beta-tubulin (deposited in GenBank under accession number AAA30261.1), T. brucei epsilon-tubulin (deposited in GenBank under accession number EAN77544.1), T. brucei TREU927 epsilon-tubulin (referenced in NCBI under numbers XP 822372.1 and XP_{—}829157.1), T. brucei delta-tubulin (deposited in GenBank under accession number EAN80045.1), T. brucei zeta-tubulin (referenced in NCBI under number XP_{—}001218818.1) or the T. brucei tubulins described in international application WO 2008/134643.

As examples of trypanosome-derived flagellar proteins, mention may be made of the T. brucei flagellar protein described in international application WO2002/19960 or the T. congolense flagellar protein described in the Applicant's French application filed on 13 Nov. 2009 under number FR09/58035. Further mention may be made of the T. brucei TREU927 flagellar protein or flagellar-like proteins (referenced in NCBI under numbers XP_{—}847376.1; XP_{—}847374.1; XP_{—}847295.1; XP_{—}843961.1; XP_{—}847377.1), the T. brucei flagellar protein TB-44A (deposited in GenBank under accession number AAZ13310.1), the T. brucei flagellar protein TB-24 (deposited in GenBank under accession number AAZ13308.1) and the T. brucei flagellar protein deposited in GenBank under accession number AAZ13311.1.

As examples of proteases, mention may be made of trypanosome cysteine proteases such as T. congolense congopain or trypanopain-Tc, T. rhodesiense rhodesain and T. cruzi chagasin or cruzipain.

The vaccines of the present invention, whether monovalent or multivalent, can further comprise adjuvants in order to increase antigenic response. Adjuvants are well-known to the person skilled in the art. As examples of adjuvants, mention may be made of vitamin E, aluminum gels or salts such as aluminum hydroxide or aluminum phosphates, metal salts, saponins, polyacrylic acid polymers such as Carbopol®, nonionic block polymers, fatty acid amines such as pyridine and DDA, dextran-based polymers such as dextran sulfate and DEAE-dextran, liposomes, bacterial immunogens such as LPS, peptidoglycans or MDP.

The nonhuman animals that can be treated include, for example, bovids, ovids, felids, suids, camelids and/or canids.

Alternatively, the vaccines can comprise an effective therapeutic amount of a monoclonal or polyclonal antibody as described below.

The multivalent vaccines of the present invention can further contain antigens of other blood parasitoses derived, for example, from protozoa such as Theileria parva, T. annulata, Babesia bigemina and B. divergens to treat and/or prevent trypanosomes and theileriosis, anaplasmosis and/or babesiosis.

These can be further combined with other standard vaccines used for the prophylaxis and/or treatment of parasitoses in the target areas, namely against foot-and-mouth disease, clostridiosis, plague, catarrhal fever, contagious bovine pleuropneumonia (CBPP), blackleg, pasteurellosis and/or sheep pox.

The vaccines of the present invention are particularly useful for treating and/or preventing trypanosomosis-induced pathogeneses such as anemia, degradations in general health, weight loss and/or immunosuppression in humans or nonhuman animals.

The monovalent or multivalent vaccines can also be administered in combination with antiparasitic agents, anti-infective agents and/or symptomatic agents.

Antiparasitic agents include, for example, trypanocidal drugs such as diamidines (pentamidine or pentamidine mesylate, diminazene or diminazene aceturate), arsenic derivatives such as Melarsoprol®, melarsomine, eflornithine (DMFO), arsobal, MelBdm, nitrofuran derivatives such as nifurtimox (5-nitrofuran), ornithine analogs (Eflornithine® or difluoromethylornithine), phenanthridine (isometamidium or Homidium®), a polysulfonated naphtha-urea such as Suramin®, an anti-malignancy agent such as quinapyramine, buthionine sulfoximine (BSO), azaserine, 6-diazo-5-oxo-norleucine (DON) and/or acivicin. When the vaccines are administered in combination with antiparasitic agents, the latter are preferably administered before and/or simultaneously and/or after the monovalent or multivalent vaccines described above. Other nonspecific antiparasitic agents for trypanosomes are well-known in the field, and are administered before and/or simultaneously and/or after the vaccines of the invention. Among these, mention may be made of avermectins (ivermectin, abamectin, doramectin, eprinomectin and selamectin), pyrethrins (deltamethrin, etc.) and/or anthelminthic antiparasitic agents (oxibendazole, piperazine, flubendazole).

As examples of anti-infective agents, mention may be made of antibiotics such as β-lactams, fosfomycin, glycopeptides or polypeptides with antibiotic activity, bacitracin, aminoglycosides, macrolides, lincosamides, streptogramins, tetracyclines, phenicols, fusidic acid or quinolones.

Symptomatic agents are, for example, anti-anemia agents such as iron, vitamin B12, folic acid or calcium levofolinate; or hepatoprotective agents such as flavonoid complexes (silymarin, silibinin, etc.), curcuma, Desmodium adscendens and/or Chrysanthellum americanum (carbon).

Non-steroidal anti-inflammatory drugs (NSAIDs) can include, among others, oxicams (meloxicam, piroxicam and/or tenoxicam), salicylate derivatives (methyl salicylate and acetylated lysine), 2-arylpropionic acids (profens), indole sulfonamide derivatives, selective COX-2 NSAIDs (celecoxib, etoricoxib, etc.), phenylbutazone, niflumic acid and/or fenamic acids.

According to a fifth aspect, the present invention relates to probes or primers specific for African trypanosome, and the use thereof in diagnostic tests.

The term “probe” as used in the present invention refers to DNA or RNA comprising at least one strand with a nucleotide sequence enabling hybridization with nucleic acids with at least one nucleotide sequence such as represented in the sequences SEQ ID NOs: 1-3, or a sequence complementary, antisense or equivalent to said sequence, and notably a sequence with five to 100 contiguous nucleotides that is at least 50%, preferably at least 60%, or at least 85% homologous to the sequences SEQ ID NOs: 1-3, or to a synthetic oligonucleotide enabling such hybridization, unmodified or comprising one or more modified bases such as inosine, methyl-5-deoxycytidine, deoxyuridine, dimethylamino-5-deoxyuridine, diamino-2,6-purine, bromo-5-deoxyuridine or any other modified base. Similarly, these probes can be modified at the sugar, namely the replacement of at least one deoxyribose with a polyamide, or at the phosphate group, for example its replacement by esters notably selected from diphosphate, dialkyl and arylphosphonate esters and phosphorothioate esters.

The probes can be much shorter than the sequences identified in the sequences SEQ ID NOs: 1-3. In practice, such probes comprise at least five nucleotides, advantageously between five and 50 nucleotides, preferably roughly 20 nucleotides, having a hybridization specificity under conditions established to form a hybridization complex with the DNA or RNA having a nucleotide sequence as previously defined. The probes of the invention can be used for diagnostic purposes, as capture and/or detection probes.

The primers of the invention comprise a sequence of five to 30 monomers selected from the sequences SEQ ID NOs: 1-3, and have a hybridization specificity under predetermined conditions to initiate enzymatic polymerization, for example in an amplification technique such as the polymerase chain reaction (PCR), in an extension process such as sequencing, in a reverse transcription method or the like.

According to a sixth aspect, the present invention relates to a detection and/or monitoring reagent as well as to a method and kits for diagnosing infections by African trypanosomes, notably by T. congolense, T. brucei, T. evansi and/or T. vivax. The trypanosome detection reagents or diagnostic kits comprise as the reactive substance at least one monoclonal or polyclonal antibody as described above. Alternatively, the trypanosome detection reagents or diagnostic kit can comprise a probe and/or primer such as defined above, to detect and/or identify African trypanosomes in a biological sample, notably a capture probe and a detection probe, with one and/or the other as defined above.

The reagent above can be bound directly or indirectly to a suitable solid support. The solid support can be notably in the form of a cone, tube, well, bead or the like. The term “solid support” as used herein includes all the materials on which a reagent can be immobilized for use in diagnostic tests. Natural or synthetic materials, chemically modified or not, can be used as solid supports, notably polysaccharides such as cellulose-based materials, for example paper, cellulose derivatives such as nitrocellulose and acetate; polymers such as vinyl chloride, polyethylene, polystyrene, polyacrylate or copolymers such as vinyl chloride and propylene polymers, vinyl chloride and vinyl acetate polymers, styrene-based copolymers, natural fibers such as cotton and synthetic fibers such as nylon.

The reagent can be bound to the solid support directly or indirectly. Directly, two approaches are possible, either by adsorption of the reagent on the solid support, i.e., by noncovalent bonds (mainly hydrogen, van der Waals or ionic bonds) or by establishment of covalent bonds between the reagent and the support. Indirectly, an “anti-reagent” compound able to interact with the reagent in order to immobilize the unit on the solid support can be bound beforehand (by adsorption or covalence) to the solid support. As an example, mention may be made of an anti-TcoTS-like 1, 2, and 3 antibody, on the condition that it is immunologically reactive with a different part of the protein than that participating in the sera antibody recognition reaction; a ligand-receptor system, for example, by grafting on the TcoTS-like 1, 2, and 3 proteins a molecule such as a vitamin, and by immobilizing the corresponding receptor on the solid phase (for example the biotin-streptavidin system). Indirect approaches also include the preliminary grafting or fusion by genetic recombination of a protein, or a fragment of said protein, or a polypeptide, at one end of the TcoTS-like 1, TcoTS-like 2 and TcoTS-like 3 proteins, and immobilization of the latter on the solid support by passive adsorption or covalence of the grafted or fused protein or polypeptide.

Capture probes can be immobilized on a solid support by any suitable means, i.e., directly or indirectly, for example by covalence or passive adsorption. Detection probes are labeled by means of a label selected from radioactive isotopes, enzymes notably selected from peroxidase and alkaline phosphatase, and those able to hydrolyze a chromogenic, fluorogenic or luminescent substrate, chemical chromophores, chromogenic, fluorogenic or luminescent compounds, nucleotide basic analogs, and biotin.

The probes of the present invention used for diagnostic purposes can be implemented in any known hybridization techniques, and notably so-called “dot-blot” techniques; Southern blot; northern blot, which is a technique identical to the Southern blot technique but which uses RNA as the target; and the sandwich technique.

The method for detecting and/or monitoring an African trypanosome infection in a biological sample, such as a blood sample from a nonhuman animal capable of being infected by African trypanosomes, consists in bringing together said sample and a reagent such as defined above, under conditions enabling a possible immunological reaction, and then detecting the presence of an immune complex with said reagent.

As a nonrestrictive example, mention may be made of the one- or multi-step ELISA detection technique, which consists in reacting a first specific monoclonal or polyclonal antibody for the antigen sought, bound to a solid support, with the sample, and revealing the possible presence of an immune complex thus formed by a second antibody labeled by any suitable label known to the person skilled in the art, notably a radioactive isotope, an enzyme, for example peroxidase or alkaline phosphatase or the like, by so-called competition techniques well-known to the person skilled in the art.

Alternatively, the method for selectively detecting African trypanosomes in a biological sample and diagnosing trypanosomosis consists in taking a blood sample, exposing the DNA extracted from the sample and optionally denaturing said DNA with at least one probe such as defined above and detecting the hybridization of said probe.

Lastly, another object of the present invention relates to a kit for veterinary use for diagnosing trypanosomiasis in a biological sample, comprising a probe or a primer as described above, or an antibody such as described above, as well as a reagent for detecting an immunological reaction.

The kits of the present invention comprise at least one compartment for an optionally sterile packaging comprising an effective therapeutic quantity of a reagent such as described above, as well as instructions relating to the protocol for implementing the veterinary diagnostics of the invention.

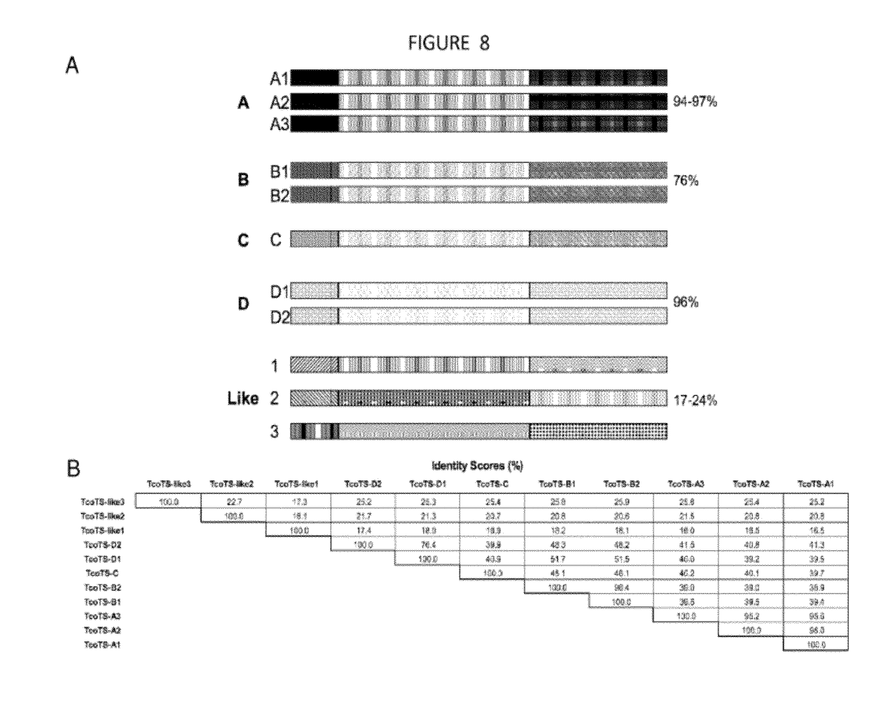

According to another aspect, the present invention concerns sequences related to trans-sialidase-like in T. congolense. More precisely, 11 genes coding for sialidase-related sequences were characterized and classified in five subfamilies according to their sequence homologies (FIGS. 8A and 8B).

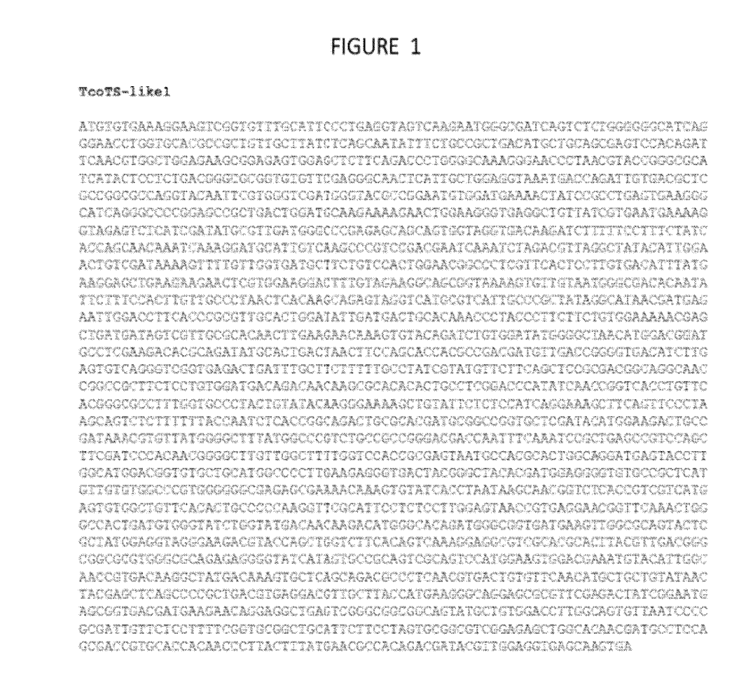

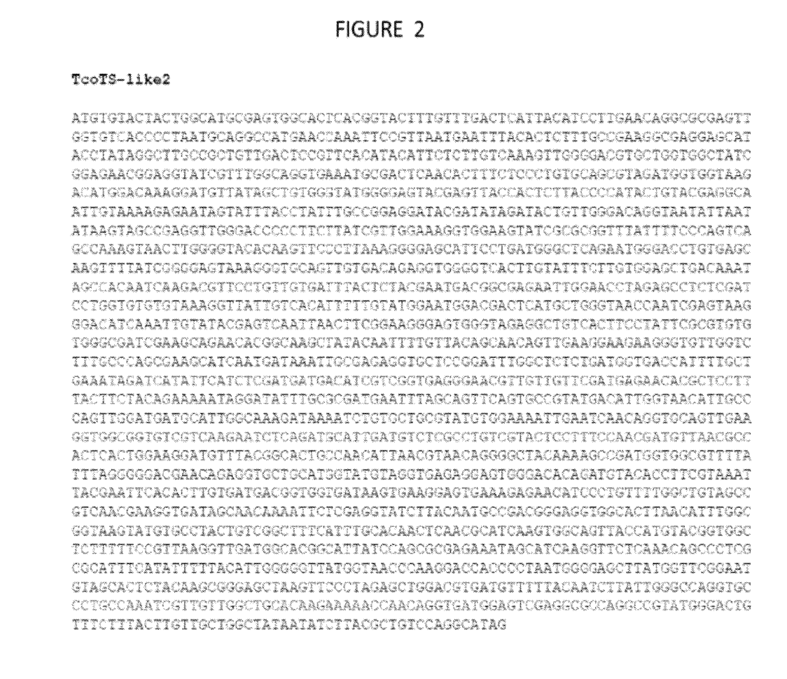

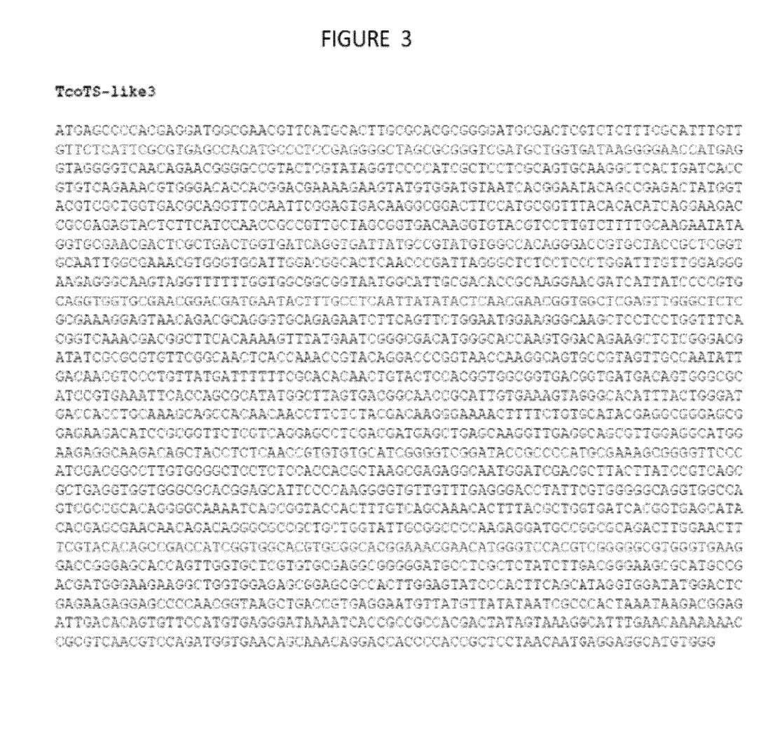

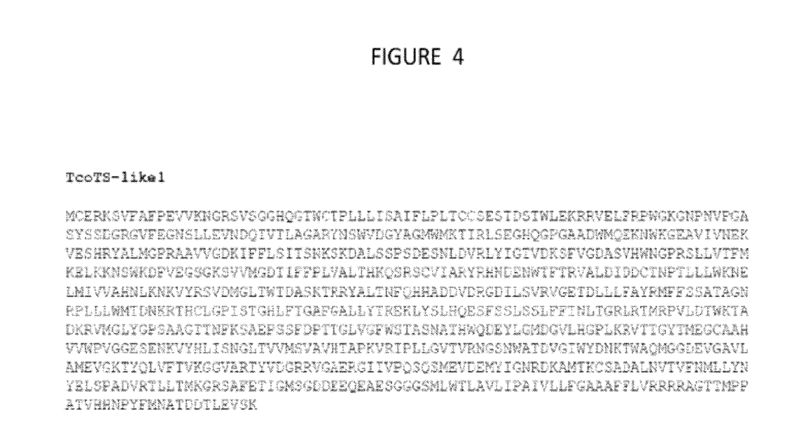

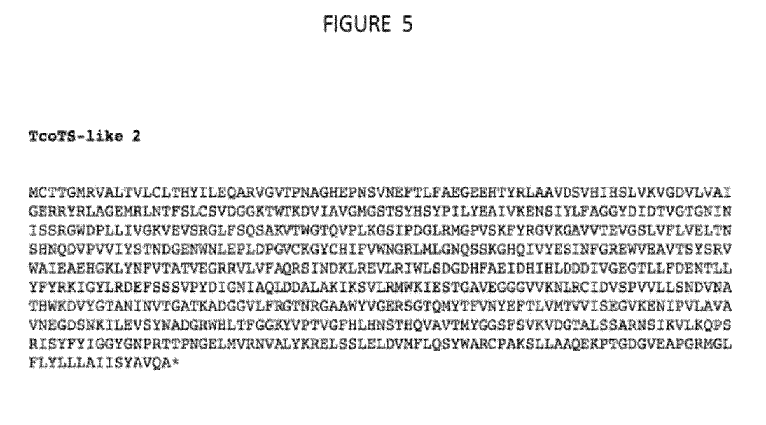

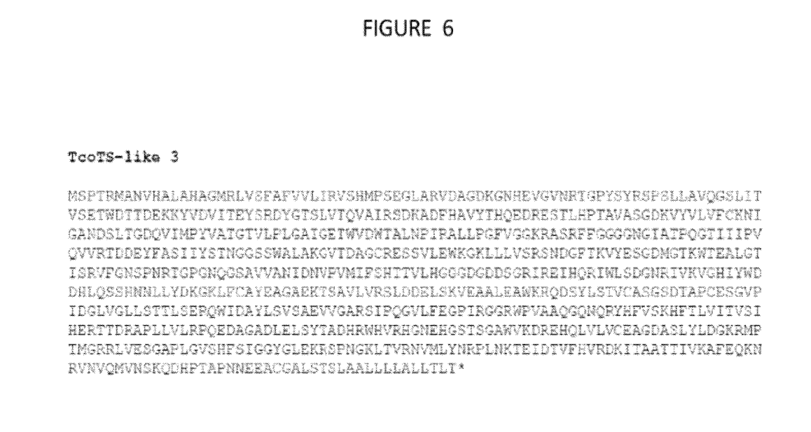

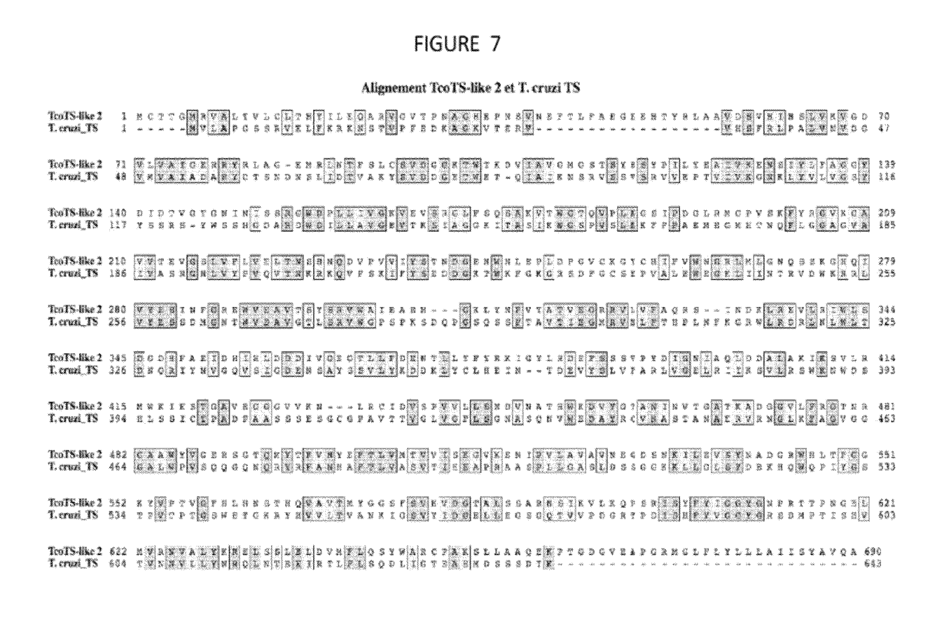

The first trans-sialidase-like subfamily comprises the three genes described above and designated TcoTS-like 1, 2 and 3, which have 17-24% identity between them (FIGS. 1 to 6).

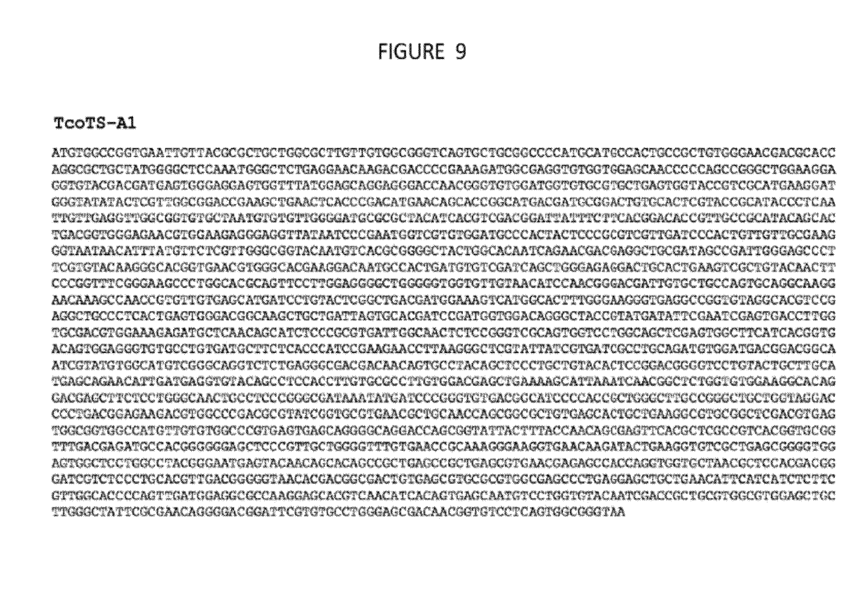

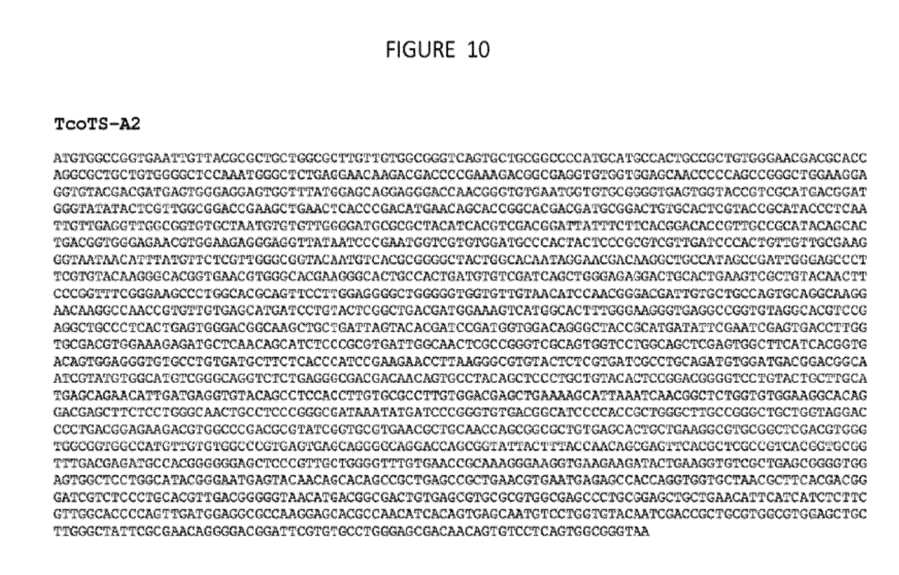

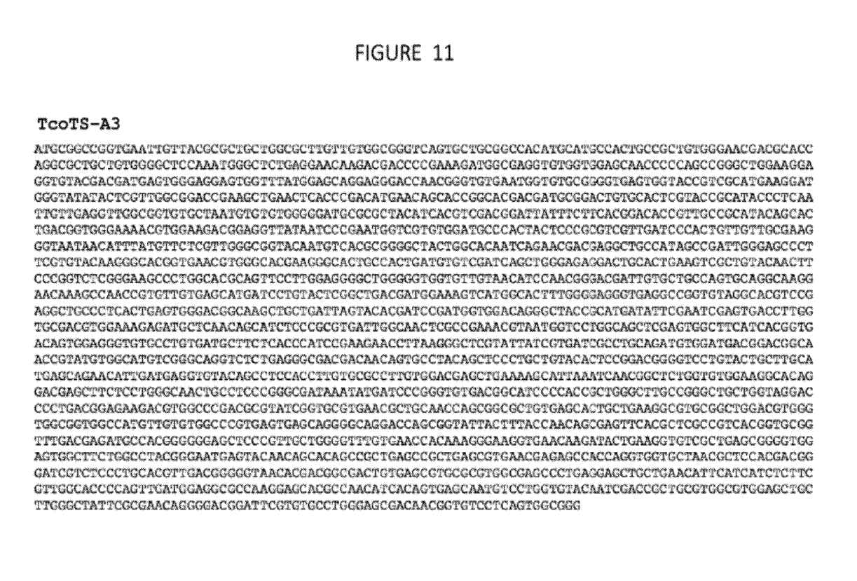

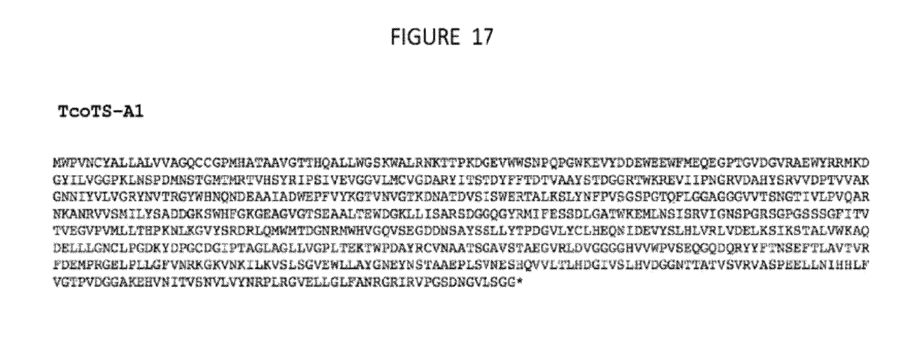

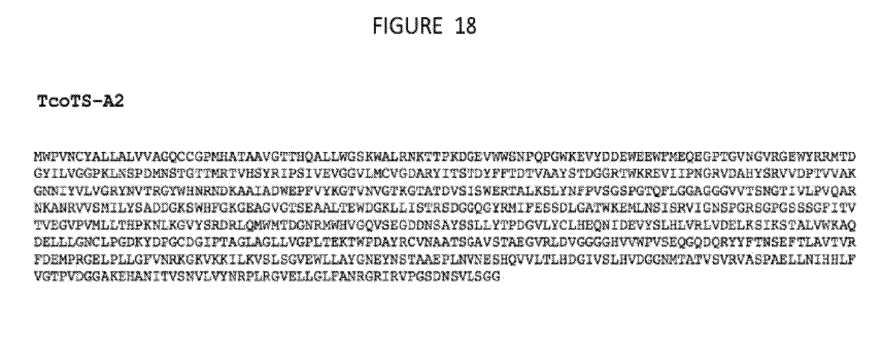

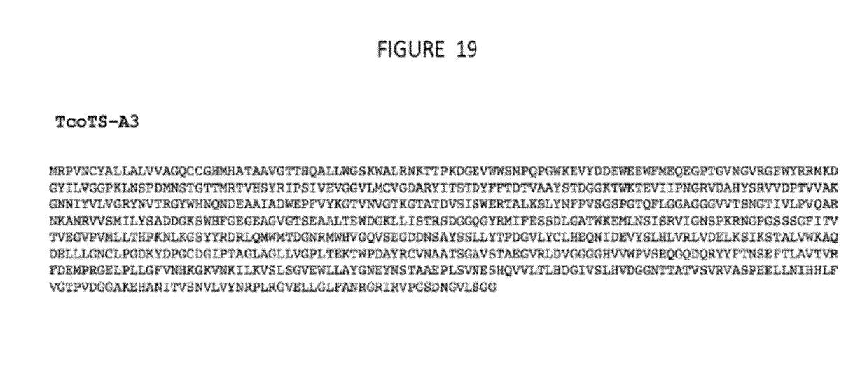

The second subfamily was named subfamily A and comprises three genes designated A1, A2 and A3 and whose nucleotide sequences are given in SEQ ID NOs: 7, 8 and 9, respectively. Genes A1, A2 and A3 have 94-97% identity between them (FIGS. 9 to 11) and code for the three proteins TcoTS-A1, TcoTS-A2 and TcoTS-A3, respectively, whose amino acid sequences are provided in SEQ ID NOs: 15, 16 and 17, respectively (FIGS. 17 to 19).

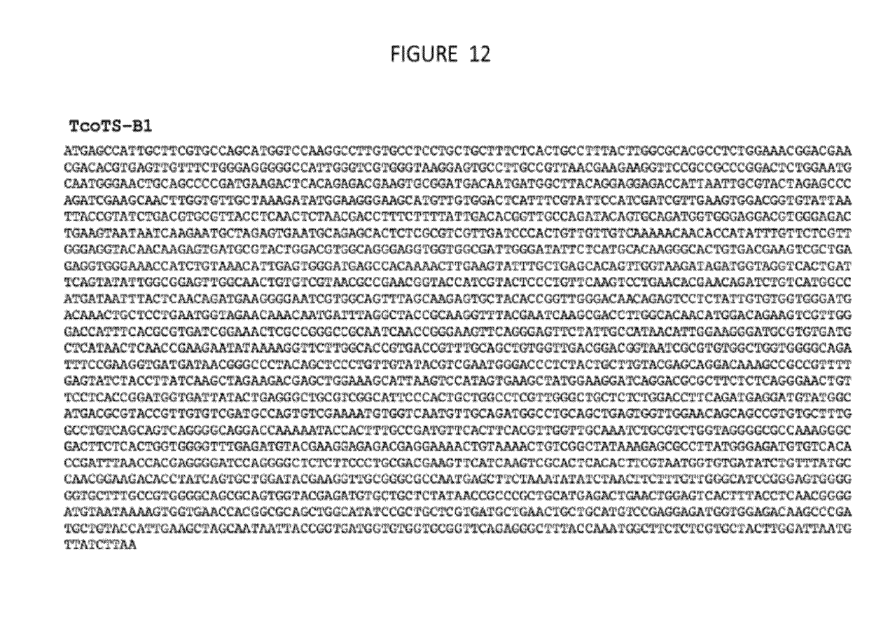

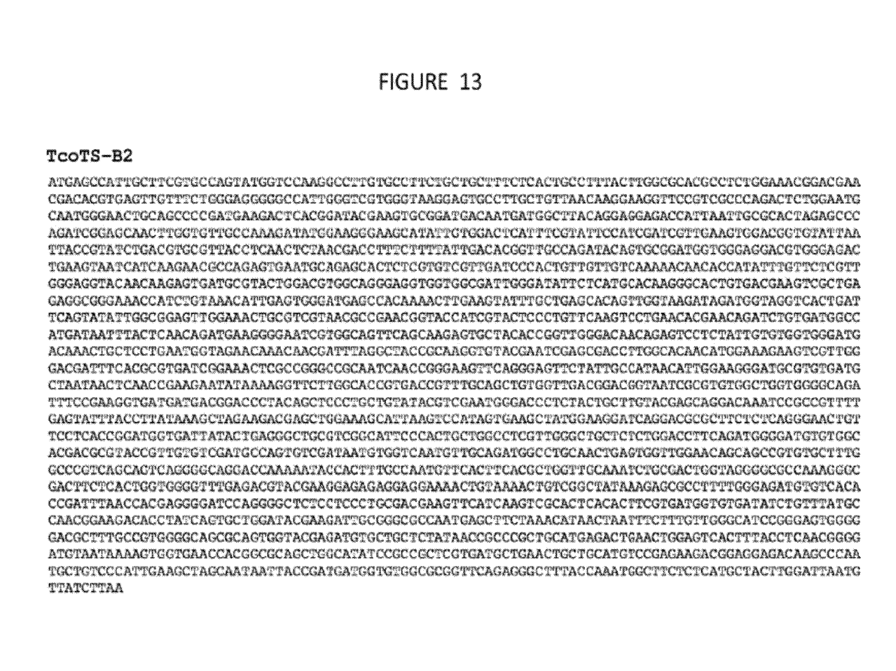

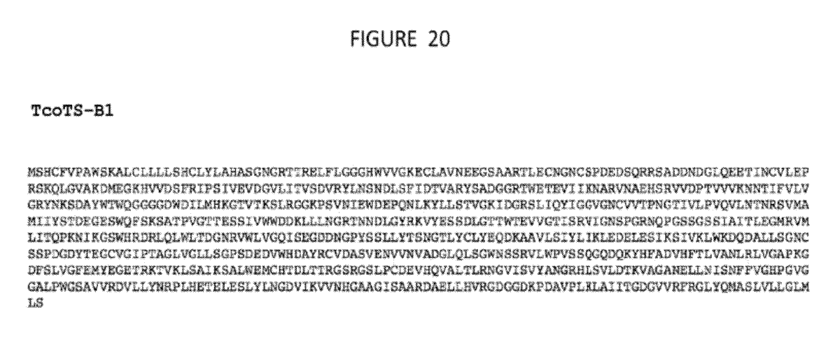

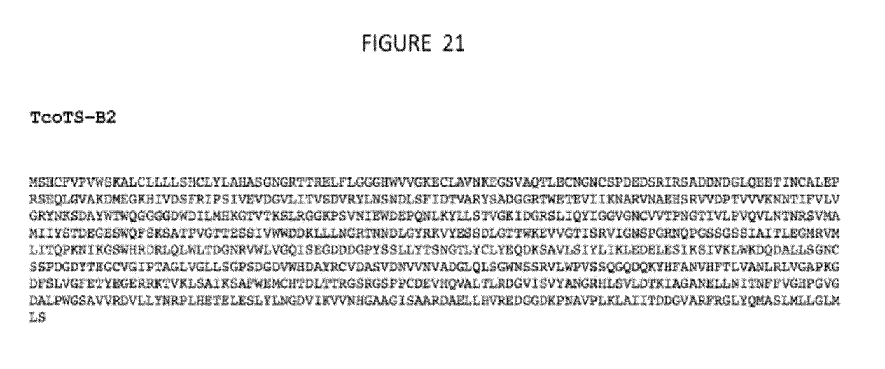

The third subfamily, designated B, comprises two genes designated hereafter B1 and B2, whose nucleotide sequences are given in SEQ ID NOs: 10 and 11, respectively, and which have 76% identity between them (FIGS. 12 and 13). The two genes B1 and B2 code for trans-sialidases TcoTS-B1 and TcoTS-B2, whose peptide sequences are represented in SEQ ID NOs: 18 and 19 (FIGS. 20 and 21).

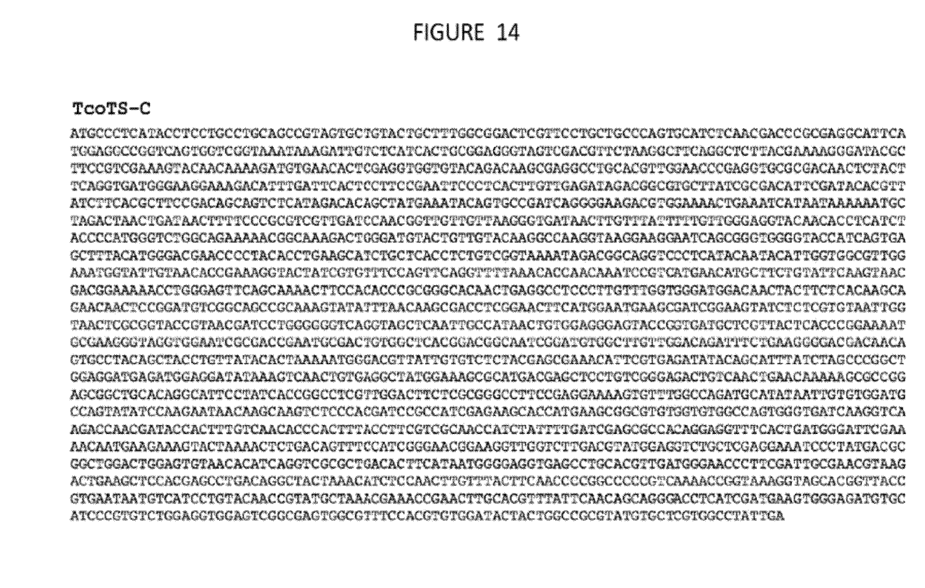

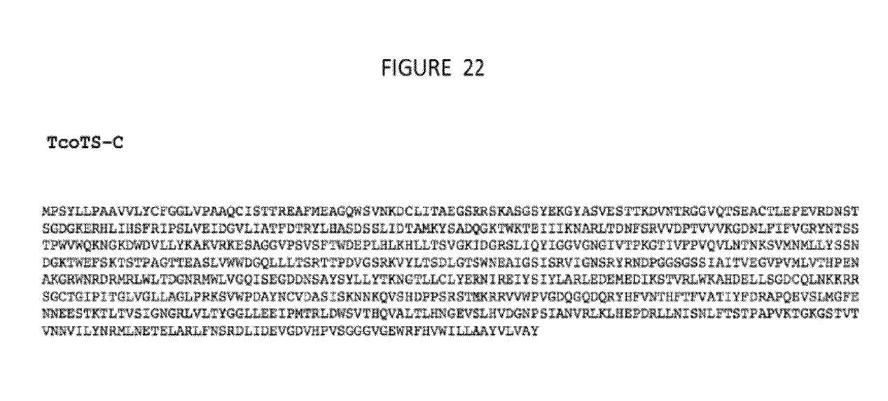

The fourth subfamily, designated C, comprises only one gene, designated C, whose nucleotide sequence is represented in SEQ ID NO: 12 (FIG. 14), and which codes for the TcoTS-C protein whose peptide sequence is provided in SEQ ID NO: 20 (FIG. 22).

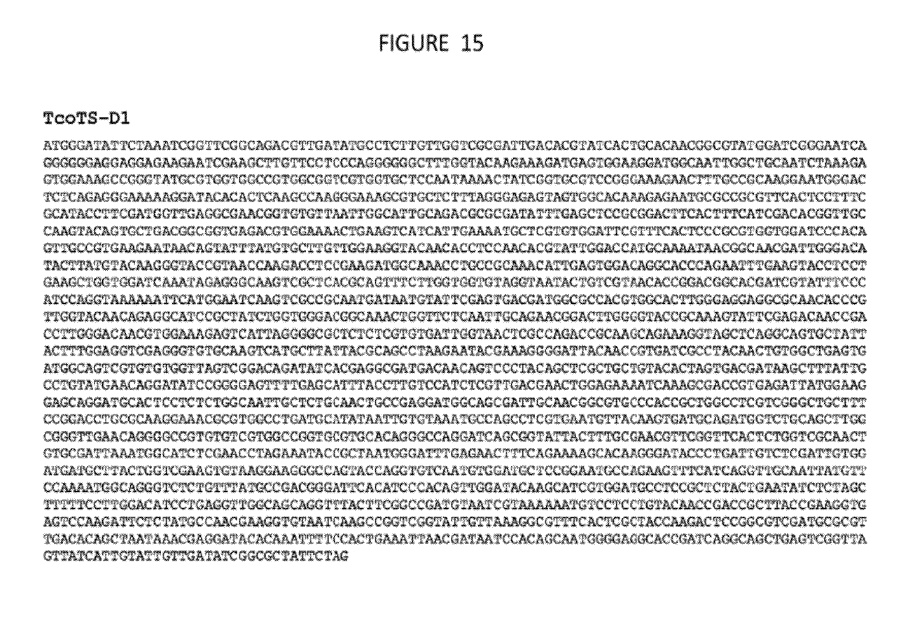

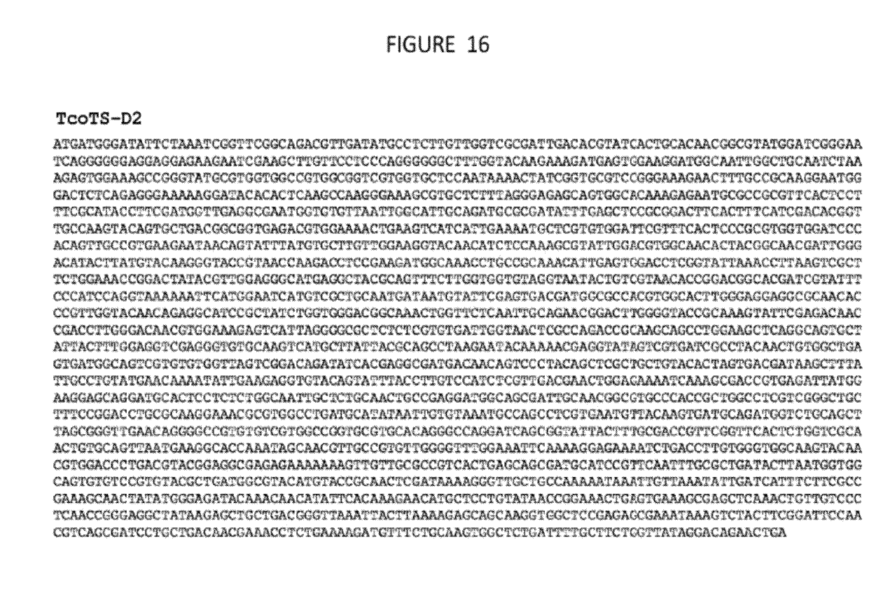

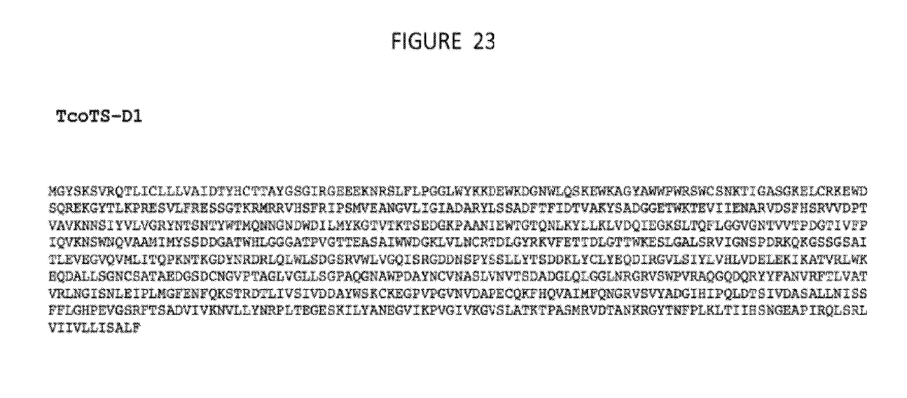

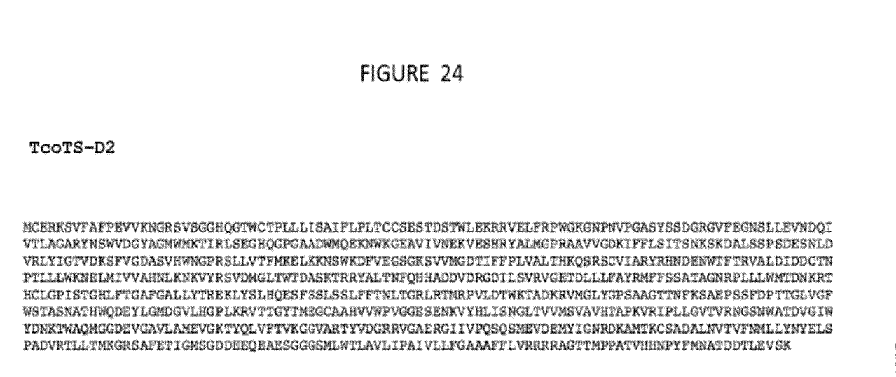

Lastly, the fifth subfamily, which was designated subfamily D, comprises two genes named D1 and D2, whose nucleotide sequences are provided in SEQ ID NOs: 13 and 14 (FIGS. 15 and 16). These two genes D1 and D2 indeed have 96% identity between them. They code for the proteins TcoTS-D1 and TcoTS-D2, whose amino acid sequences are provided in SEQ ID NOs: 21 and 22 (FIGS. 23 and 24).

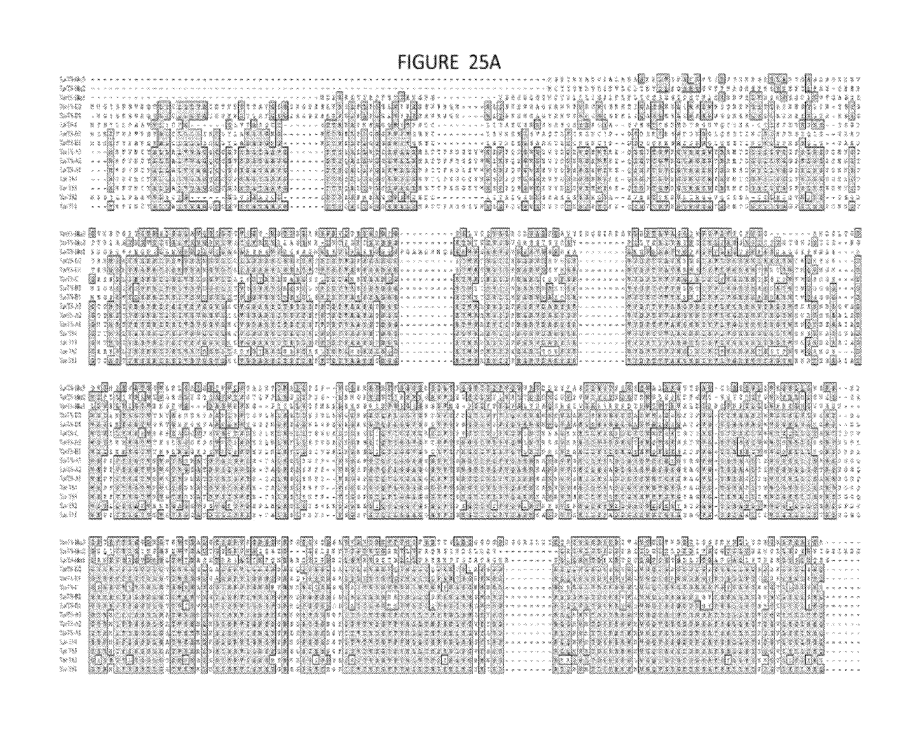

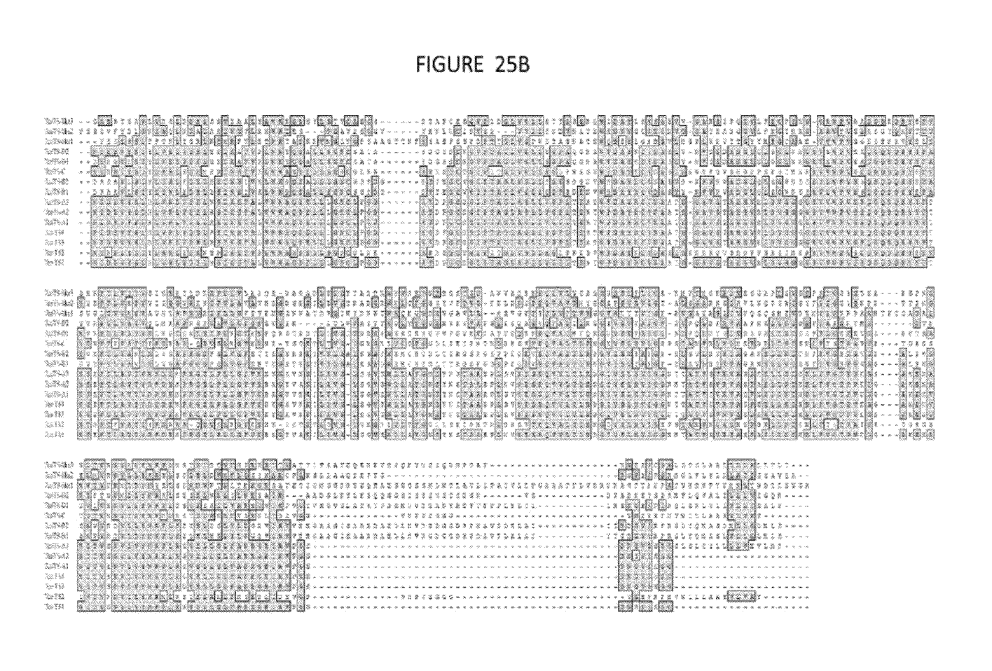

The percent identities between the proteins coded by these 11 genes of the invention as described above are presented in FIGS. 8A and 8B. An alignment of the sequences is given in FIGS. 25A and 25B. Trans-sialidase-like 1 to 3 are highly divergent in relation to other genes.

According to this aspect, the present invention thus has as an object novel nucleotide sequences, coding for novel trans-sialidase-like proteins, called TcoTS-A1, TcoTS-A2, TcoTS-A3, TcoTS-B1, TcoTS-B2, TcoTS-C, TcoTS-D1 and TcoTS-D2 belonging to African trypanosomes. These novel DNA or RNA molecules comprise at least one strand comprising a nucleotide sequence selected from the sequences SEQ ID NOs: 7-14, a sequence complementary, antisense or equivalent to one of the sequences SEQ ID NOs: 7-14, and notably a sequence comprising an identity of at least 70% with one of the sequences SEQ ID NOs: 7-14, or a sequence having, on a sequence of 100 contiguous nucleotides, at least 50%, preferably at least 60%, or at least 70%, or at least 80% homology with said sequences, or a nucleotide sequence able to hybridize with one of the sequences SEQ ID NOs: 7-14 under stringent hybridization conditions, such as defined above.

The invention also relates to DNA or RNA fragments whose nucleotide sequence is identical, complementary, antisense or equivalent to any of the sequences SEQ ID NOs: 7-14, and notably DNA or RNA fragments, for any sequence of 30 contiguous monomers, at least 50%, preferably at least 60%, or at least 85% homologous to any one of said sequences.

Also, according to this aspect, the invention relates to proteins called TcoTS-A1, TcoTS-A2, TcoTS-A3, TcoTS-B1, TcoTS-B2, TcoTS-C, TcoTS-D1 and TcoTS-D2, as well as the peptide sequences of said proteins as represented in the sequences SEQ ID NOs: 15-22, respectively, and all amino acid sequences having a homology of at least 70%, 75%, 80%, 85%, 90%, or at least 95% with the peptide sequences SEQ ID NOs: 15-22. The invention also has as an object all antigenic peptide fragments of the proteins TcoTS-A1, TcoTS-A2, TcoTS-A3, TcoTS-B1, TcoTS-B2, TcoTS-C, TcoTS-D1 and TcoTS-D2 specifically recognized by anti-African trypanosome antisera, as well as all immunological functional equivalents of said proteins likely to be recognized immunologically by antibodies directed against the proteins TcoTS-A1, TcoTS-A2, TcoTS-A3, TcoTS-B1, TcoTS-B2, TcoTS-C, TcoTS-D1 and TcoTS-D2 of the present invention. Said proteins and antigenic peptide fragments of the invention can comprise modifications, notably chemical modifications that do not deteriorate their immunogenicity.

As an example, an antigenic peptide fragment of the present invention can be the peptide PKNIKGSWHRDRLQLWLTD (SEQ ID NO: 24) belonging to the TcoTS-B1 protein or peptides at least 70%, 75%, 80%, 85%, 90%, or at least 95% homologous to said fragment.

The present invention further relates to the combination or a mixture of one or more proteins selected from TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2, and/or one or more antigenic peptide fragments of said proteins, and/or one or more immunological functional equivalents of said proteins. Also, it has as an object a method for preparing one or more proteins selected from TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2, or a mixture of said proteins, and/or one or more antigenic peptide fragments of said proteins, and/or one or more immunological functional equivalents of said proteins. These techniques for producing proteins, fragments, functional equivalents and combinations are carried out by chemical synthesis, protein lysis or genetic recombination. They are well-known to the person skilled in the art, and have been in addition described above.

According to this aspect, the invention relates to monoclonal or polyclonal antibodies obtained by immunological reaction of a nonhuman animal organism with an immunogenic agent comprised of one or more natural or recombinant TcoTS-A1, TcoTS-A2, TcoTS-A3, TcoTS-B1, TcoTS-B2, TcoTS-C, TcoTS-D1 and TcoTS-D2 proteins and the peptide fragments thereof such as described above. It also has as an object a vaccine composition comprising a mixture of one or more proteins selected from TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2, and/or one or more antigenic peptide fragments of said proteins, and/or one or more immunological functional equivalents of said proteins and/or a combination of said proteins, fragments or functional equivalents.

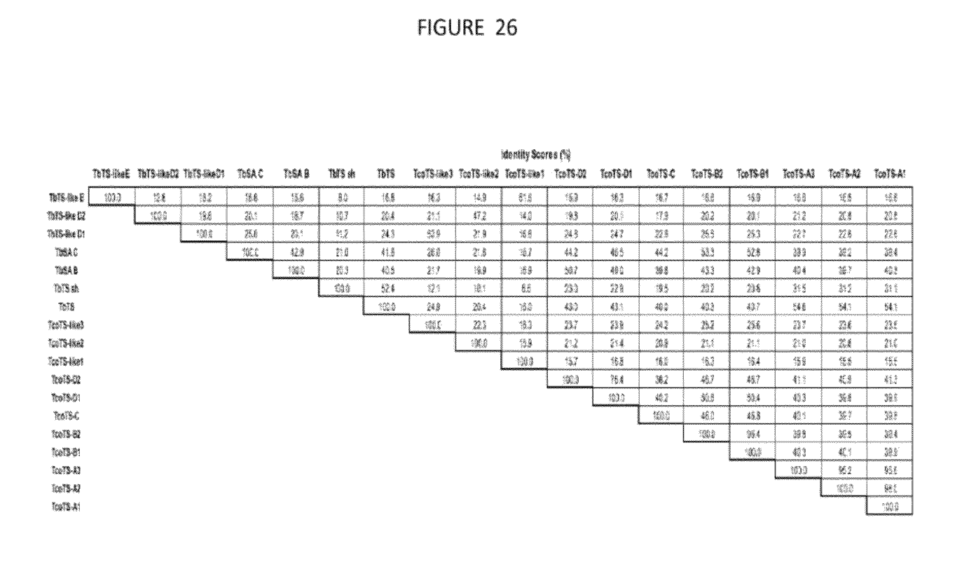

Up to the present, none of these 11 proteins has been identified in the blood forms of T. congolense. Indeed, Tiralongo et al. ((2003) J. Biol. Chem. 278(26):23301-10) as well as the international publication WO2004/055176 describe the cloning in the procyclic forms present in the insect vector of two T. congolense trans-sialidases, TS1 and TS2. Said proteins were only described as being expressed in the procyclic forms present in the insect vector. Also, a study of sialidase-related genes was carried out in T. brucei (Montagna et al. (2006) J. Biol. Chem. 281(45): 33949-58). Montagna et al. describe the identification of several protein sequences of the T. brucei TbTS gene family (AF310231.1). It notably describes a truncated version of the TbTS gene, namely TbTSsh, the genes B and C coding for T. brucei trans-sialidases TbSA B and TbSA C, and finally the genes D1, D2, and E coding for T. brucei trans-sialidases. The percent identities between the sequences identified in T. congolense and T. brucei are presented in FIG. 26. Montagna et al. disclose that these trans-sialidases are expressed in vivo in the procyclic forms or insect forms, and likely play an important role in the transfer of sialic acid on the parasite membrane, thus ensuring the protection of the parasites and their survival when they are transported by insect vectors. However, Montagna et al. do not describe the possibility of detecting these trans-sialidases in sufficient quantity in the blood forms of parasites, i.e., in the infected animals, and thus using them as vaccines or diagnostics.

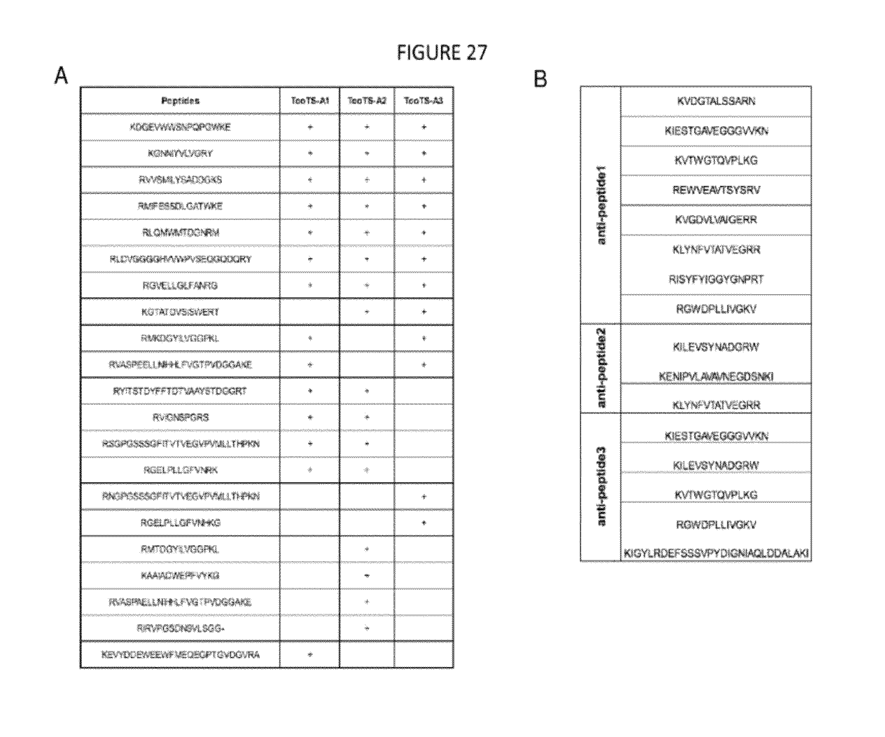

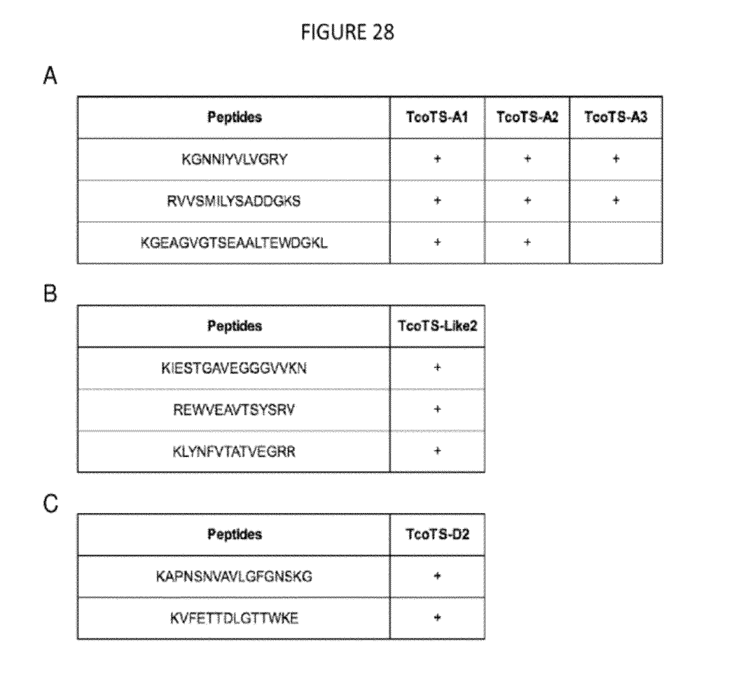

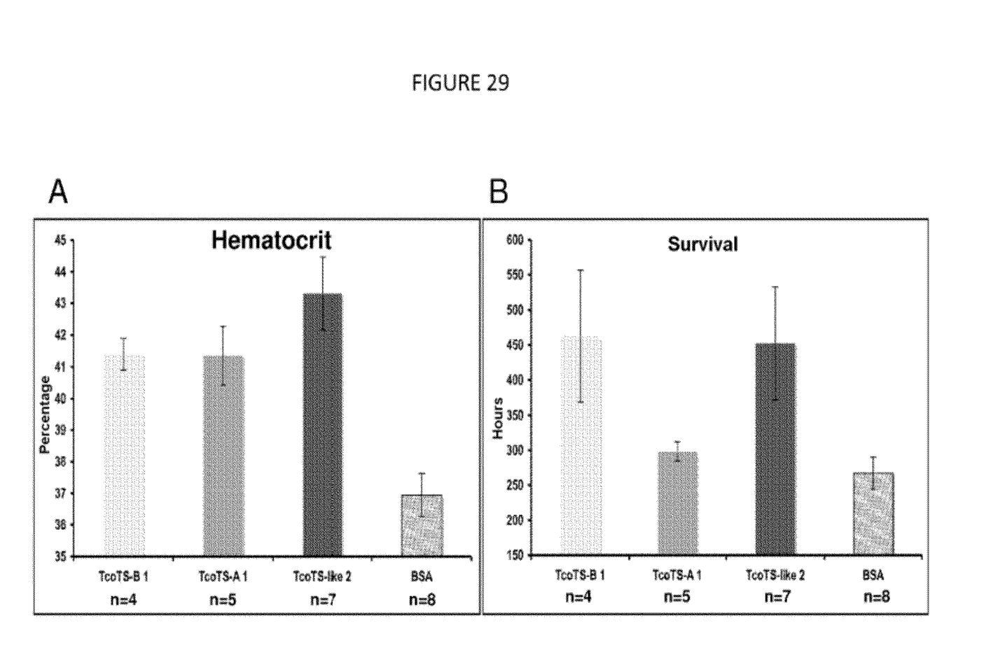

Also, up to the present no sialidase activity has been described for these 11 proteins in blood forms. On the contrary, the literature describes the absence of sialidase activity in T. congolense blood forms (Engstler et al. (1995) Acta Trop. 59: 117-29).

Whereas none of these 11 proteins has ever been identified in T. congolense blood forms and no sialidase activity has been described in these forms, the Applicant has demonstrated in a surprising manner sialidase activity in T. congolense blood forms, and has shown by immunoprecipitation followed by mass spectrometry analysis the expression of TcoTS-A1, TcoTS-A2, TcoTS-A3 and TcoTS-like 2 proteins in T. congolense blood forms (Example 3 and FIG. 27). The expression of these same proteins as well as the TcoTS-D2 protein was also shown by mass spectrometry analysis of T. congolense blood form membrane preparations (Example 4 and FIG. 28). The applicant further demonstrated during vaccination protection experiments on murine models (Example 5, FIGS. 29A and 29B) that the antigenic proteins TcoTS-A1, TcoTS-B1 and TS-like 2 produced a greater protective effect in terms of mean survival of the animals as well as in relation to hematocrit. This protection was even total (no development of parasitemia and normal hematocrit) in certain cases: three mice out of 12 in the case of TcoTS-like 2 and one out of nine in the case of TcoTS-B1.

Consequently, the present invention has as an object vaccine or veterinary compositions intended to treat and/or prevent an African trypanosome infection in a nonhuman animal, particularly against infections by the species T. congolense, T. brucei, T. evansi and/or T. vivax. Said veterinary vaccine compositions can be provided in the form of an antigenic vaccine and thus comprise a therapeutically effective quantity of one or more proteins selected from TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2, and/or one or more antigenic peptide fragments of said proteins, and/or one or more immunological functional equivalents of said proteins and/or a combination of said proteins, fragments or functional equivalents. Preferably, said vaccine or veterinary compositions comprise at least one protein selected from TcoTS-A1, TcoTS-B1 and TcoTS-like 2. Even more preferentially, said vaccine or veterinary compositions comprise at least the TcoTS-like 2 protein, and/or an antigenic peptide fragment, and/or an immunological functional equivalent of TcoTS-like 2. Alternatively, the vaccine compositions can comprise an effective therapeutic quantity of a monoclonal or polyclonal antibody directed against one or more proteins selected from TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2. They are particularly useful for treating and/or preventing trypanosomosis-induced pathogeneses, notably such as anemia, degradations in general health, weight loss and/or immunosuppression in nonhuman animals.

Further according to this aspect, the present invention relates to a reagent for detecting and/or monitoring as well as a method and kits for diagnosing African trypanosome infections, notably by T. congolense, T. brucei, T. evansi and/or T. vivax. The trypanosome detection reagents or diagnostic kits comprise as the reactive substance at least one monoclonal or polyclonal antibody directed against one or more TcoTS-A1, TcoTS-A2, TcoTS-A3, TcoTS-B1, TcoTS-B2, TcoTS-C, TcoTS-D1 and TcoTS-D2 proteins. Preferably, the trypanosome detection reagents or diagnostic kits comprise as the reactive substance at least one monoclonal or polyclonal antibody directed against one or more proteins selected from TcoTS-A1, TcoTS-A2, TcoTS-A3 and TcoTS-like 2.

The method for detecting and/or monitoring an African trypanosome infection in a biological sample, such as a blood sample from a nonhuman animal able to be infected by African trypanosomes, consists in bringing together said sample and a reagent such as defined above, under conditions enabling a possible immunological reaction, and then detecting the presence of an immune complex with said reagent.

As a nonrestrictive example, mention may be made of the one- or multi-step ELISA detection technique, which consists in reacting a first specific monoclonal or polyclonal antibody for the antigen sought, bound to a solid support, with the sample, and revealing the possible presence of an immune complex thus formed by a second antibody labeled by any suitable label known to the person skilled in the art, notably a radioactive isotope, an enzyme, for example peroxidase or alkaline phosphatase or the like, by so-called competition techniques well-known to the person skilled in the art.

Finally, according to this aspect, the present invention has as an object a kit for veterinary use for diagnosing trypanosomosis in a biological sample, comprising an antibody such as described above as well as a reagent for detecting an immunological reaction. The kits of the present invention comprise at least one compartment for an optionally sterile packaging comprising an effective therapeutic quantity of a reagent such as described above, as well as instructions relating to the protocol for implementing the veterinary diagnostics of the invention.

### EXAMPLES

### Example 1

### Production of Polyclonal Antibodies Directed Against the TcoTS-A1 Protein

The TcoTS-A1 protein was produced in the yeast Pichia pastoris. To that end, the X33 strain was transformed by the PICZ vector (Invitrogen) containing the sequence coding for the TcoTS-A1 protein lacking its first 29 amino acids. The protein secreted in the culture supernatant after 4 days of expression induction in methanol was purified by successive ion-exchange chromatographies. First, the culture supernatant was dialyzed against 20 mM Na acetate buffer (pH 4.5) for 16 hours, centrifuged for 30 minutes at 10,000 g, and then subjected to chromatography on one 1 ml HiTrap SP HP column (GE Healthcare). Elution was carried out according to a linear gradient of 0-1 M NaCl. Fractions containing sialidase activity (fluorometry test with the substrate 2′-(4-methylumbelliferyl)-α-D-N-acetylneuraminic acid, as described in the publication by Montagna et al. (2006) J. Biol. Chem. 281(45): 33949-58), were combined and dialyzed for 16 hours against 20 mM Tris-HCl buffer (pH 8). After centrifugation for 30 minutes at 10,000 g, the supernatant was subjected to a second chromatography on one 1 ml HiTrap Q HP column (GE Healthcare). Elution was carried out according to a linear gradient of 0-1 M NaCl. The fractions containing sialidase activity were combined and treated with the endoglycosidase Endo Hf (Biolabs) according to the manufacturer's recommendations. The deglycosylated sample was again subjected to chromatography on one 1 ml HiTrap Q HP column (GE Healthcare) as described above. Protein integrity was verified by SDS-PAGE and staining with Coomassie blue.

This purified recombinant protein was then used to immunize BALB/c mice or rabbits. 20 μg of recombinant protein was injected into mice on a schedule of one injection each 15 days for a total of four injections or 100 μg of recombinant protein was injected into rabbits on a schedule of one injection each 15 days for a total of four injections. For the first injection, the recombinant protein was mixed in emulsion form with Freund's complete adjuvant and then for the following injections with Freund's incomplete adjuvant. Serum from the immunized animals was collected at the end of the experiment (anti-TcoTS-A1 serum) and its reactivity against the recombinant protein was verified by indirect ELISA.

### Example 2

### Production of Polyclonal Antibodies Directed Against Peptides from Sialidase-Related Sequences

The following peptides: C-RTSIDYHLIDTVAKYSADDG (SEQ ID NO: 23), C-PKNIKGSWHRDRLQLWLTD (SEQ ID NO: 24) and C-PVSAQGQDHRYEAANAEHT (SEQ ID NO: 25), named peptides 1, 2 and 3, respectively, were coupled via the N-terminus cysteine with a carrier protein (KLH) activated by a maleimide functional group and used to immunize rabbits on a schedule of one 100 μg injection every 20 days for a total of five injections. For the first injection, the recombinant protein was mixed in emulsion form with Freund's complete adjuvant and then for the following injections with Freund's incomplete adjuvant. The polyclonal sera obtained, designated anti-peptide 1 antibody, anti-peptide 2 antibody and anti-peptide 3 antibody, respectively, were collected at the end of the experiment and verified for their reactivity against their respective peptide by indirect ELISA.

### Example 3

### Demonstration of TcoTS-A1, TcoTS-A2, TcoTS-A3 and TcoTS-Like 2 Protein Expression in T. congolense Blood Forms

3 ml of rabbit serum or 1 ml of mouse serum was dialyzed against 1 l of 20 mM phosphate buffer (pH 7) for 16 hours. The dialyzed serum was centrifuged for 20 minutes at 5,000 g and then passed through one protein G sepharose Fast Flow column (GE healthcare) prepared beforehand as indicated by the manufacturer. After washing the column with 20 mM phosphate buffer (pH 7), the IgG bound to the column were eluted with 0.1 M glycine HCl buffer (pH 2.6). The IgG thus purified were dialyzed for 16 hours against 1 l of 0.1 M NaHCO₃ (pH 8.3)/0.5 M NaCl buffer. The IgG were then incubated for 2 hours at room temperature with CNBr-activated sepharose (Sigma) prepared beforehand according to the manufacturer's recommendations. After centrifugation for 1 minute at 1,000 g, the resin was washed with the previous buffer and then saturated by adding 0.1 M Tris-HCl (pH 8) for 2 hours at room temperature. After centrifugation for 1 minute at 1,000 g, the resin was washed successively with Tris-HCl (pH 8)/0.5 M NaCl buffer and then 0.1 M Na acetate (pH 4)/0.5 M NaCl buffer. The resin thus prepared for use in an immunoprecipitation experiment was equilibrated with OLB (100 mM KCl, 17% glycerol, 1 mM MgCl₂, 2.25 mM CaCl₂, 0.5% NP40, 10 mM Tris-HCl, pH 8). 10⁹ cells of the IL3000 strain were lysed in OLB for 1 hour at 4° C. and then centrifuged for 10 minutes at 20,000 g. The supernatant was incubated with the resin prepared beforehand for 16 hours at 4° C. The resin was then centrifuged for 1 minute at 1,000 g and then rinsed with OLB. The antigens bound to the IgG were eluted with 2% boiling SDS. The eluate was dialyzed against water and then freeze-dried. The lyophilizate was then taken up in Laemmli buffer (50 mM Tris-HCl (pH 6.8), 10% glycerol, 1% SDS, 2.5% γ-mercaptoethanol, 0.01% bromophenol blue) and then subjected to SDS PAGE. The gel was then stained with silver nitrate and the bands thus revealed were cut out and analyzed using tandem mass spectrometry (MS/MS).

This protocol was carried out with anti-TcoTS-A1, anti-peptide 1, anti-peptide 2 and anti-peptide 3 polyclonal sera on the procyclic forms and the blood forms of the IL3000 strain of T. congolense. The results for the blood forms are presented in FIG. 27. Immunoprecipitation with the anti-TcoTS-A1 serum identified TcoTS-A1, TcoTS-A2 and TcoTS-A3 proteins in the T. congolense procyclic forms and blood forms. Immunoprecipitations with the anti-peptide 1, anti-peptide 2 and anti-peptide 3 sera identified TcoTS-like 2 protein only in T. congolense blood forms. These results demonstrated for the first time the expression of TcoTS-A1, TcoTS-A2, TcoTS-A3 and TcoTS-like 2 proteins in the blood forms of the parasite.

### Example 4

### Demonstration of TcoTS-A1, TcoTS-A2, TcoTS-A3, TcoTS-Like 2 and TcoTS-D2 Protein Expression in T. congolense Blood Form Membrane Preparations

10⁹ cells of the IL3000 strain were lysed in 1 ml of hypotonic buffer (5 mM Na₂HPO₄, 0.3 mM KH₂PO₄) for 30 minutes at 4° C. and then centrifuged for 10 minutes at 20,000 g. The pellet was subjected to the same treatment three times in a row. The last pellet is taken up at 4° C. in 100 μl of this same hypotonic lysis buffer to which is then added 0.5 ml of the following buffer: 2 mM EDTA, 15.4 mM NaOH, 0.2 mM dithiothreitol. After 10 minutes of incubation, the mixture is centrifuged for 10 minutes at 20,000 g. The supernatant is recovered (soluble fraction) and the pellet (insoluble fraction) is taken up in 50 μl of water to which is then added 50 μl of 2% SDS. 50 μl of each of these two fractions are mixed with 15 μl of 4× Laemmli buffer (200 mM Tris-HCl pH 6.8, 40% glycerol, 4% SDS, 10% γ-mercaptoethanol, 0.04% bromophenol blue) heated at 100° C. for 10 minutes and then subjected to SDS-PAGE. The gel was then stained with silver nitrate and the bands thus revealed were cut out and analyzed using tandem mass spectrometry (MS/MS).

### Example 5

### Vaccination Tests on a Murine Model

### Example 5.1

### Vaccination Tests with TcoTS-like 1

Two groups of BALB/c mice were injected intraperitoneally with either 20 μg of BSA (negative control group) or recombinant TcoTS-like 1 protein (immunized mice group) on a schedule of one injection each 15 days for a total of four injections. Then, the mice were infected with 10⁴ parasites of T. congolense strain IL3000. Hematocrit and parasitemia were measured every 2 days for both groups of mice.

### Example 5.2

### Vaccination Tests with TcoTS-Like 2

Fourteen BALB/c type mice were injected intraperitoneally with 20 μg of BSA (7 negative control mice) or recombinant TcoTS-like 2 protein (7 mice) on a schedule of one injection each 15 days for a total of four injections. Then, the mice were infected with 10⁴ parasites of T. congolense strain IL3000. Hematocrit and parasitemia were measured every 2 days. Mean hematocrit over the entire duration of the parasitemia was calculated: it is 43.3±1.2% for the mice immunized with TcoTS-like 2 and 37.0±0.7% for the control mice immunized with BSA (FIG. 28).

Mean survival of the mice was also determined: it is 453±81 hours for the mice immunized with TcoTS-like 2 and 267±23 hours for the control mice immunized with BSA.

### Example 5.3

### Vaccination Tests with TcoTS-like 3

Two groups of BALB/c mice were injected intraperitoneally with either 20 μg of BSA (negative control group) or recombinant TcoTS-like 3 protein (immunized mice group) on a schedule of one injection each 15 days for a total of four injections. Then, the mice were infected with 10⁴ parasites of T. congolense strain IL3000. Hematocrit and parasitemia were measured every 2 days for both groups of mice.

### Example 5.4

### Vaccination Tests with TcoTS-A1

Thirteen BALB/c mice were injected intraperitoneally with either 20 μg of BSA (8 negative control mice) or recombinant protein TcoTS-A1 (5 mice) on a schedule of one injection each 15 days for a total of four injections. Then, the mice were infected with 10⁴ parasites of T. congolense strain IL3000. Hematocrit and parasitemia were measured every 2 days. Mean hematocrit over the entire duration of the parasitemia was calculated: it is 41.4±0.9% for the mice immunized with TcoTS-A1 and 37.0±0.7% for the control mice immunized with BSA (FIG. 28).

Mean survival of the mice was also determined: it is 299±14 hours for the mice immunized with TcoTS-A1 and 267±23 hours for the control mice immunized with BSA.

### Example 5.5

### Vaccination Tests with TcoTS-B1

Twelve BALB/c mice were injected intraperitoneally with either 20 μg of BSA (8 negative control mice) or recombinant protein TcoTS-B1 (4 mice) on a schedule of one injection each 15 days for a total of four injections. Next, the mice were infected with 10⁴ parasites of T. congolense strain IL3000. Hematocrit and parasitemia were measured every 2 days.

Mean hematocrit over the entire duration of the parasitemia was calculated: it is 41.4±0.5% for the mice immunized with TcoTS-B1 and 37.0±0.7% for the control mice immunized with BSA (FIG. 28).

Mean survival of the mice was also determined: it is 463±94 hours for the mice immunized with TcoTS-B1 and 267±23 hours for the control mice immunized with BSA.

### Example 5.6

### Vaccination Tests with One or More Proteins Selected from TcoTS-A2, TcoTS-A3, TcoTS-B2, TcoTS-C, TcoTS-D1 and TcoTS-D2

Two groups of BALB/c mice were injected intraperitoneally with either 20 μg of BSA (negative control group) or one or more recombinant proteins selected from the proteins TcoTS-A2, TcoTS-A3, TcoTS-B2, TcoTS-C, TcoTS-D1 and TcoTS-D2 (immunized mice group) on a schedule of one injection each 15 days for a total of four injections. Next, the mice were infected with 10⁴ parasites of T. congolense strain IL3000. Hematocrit and parasitemia are measured every 2 days for both groups of mice.

### Example 6

### Vaccination Tests on Cattle

Two groups of cattle were injected subcutaneously with one or more antigens such as TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2, mixed with two types of adjuvants, 1 mg/ml Quil A (saponin) and AdjuPhos (colloidal aluminum phosphate) volume to volume according to a final volume of 1 ml or just with the adjuvant mixture (control). One injection was given each three weeks for a total of three injections of 100 μg, 50 μg and 25 μg of antigen, respectively. The animals were infected by T. congolense strain IL3000 three weeks after the last injection in a ratio of 1,000 parasites per animal intradermally. Blood samples were taken daily until all the animals were recognized as infected, parasitemia being determined by buffy-coat analysis. Thereafter, weekly blood samples were taken to monitor parasitemia and anemia, and the animals were weighed monthly. The kinetics of the response to immunization and to infection were monitored by ELISA on the various immunizing antigens.

The antigens used during this immunization experiment were TcoTS-like 1, 2 or 3 or TcoTS-A1 or TcoTS-B1, alone or in one of all possible combinations.

### Example 7

### Example of Diagnostic Tests on Infected Animal Blood

This test is carried out by detecting circulating antigens such as TcoTS-A1, TcoTS-A2, TcoTS-A3 and TcoTS-like 2 by the sandwich ELISA method. The so-called capture antibody is adsorbed in the wells of a 96-well plate by incubation overnight at 4° C. of 1-10 μg/ml of capture antibody diluted in 100 μl of 50 mM NaHCO₃ buffer (pH 9.6). The plate is then emptied and washed three times with 200 μl per well of PBS-Tween solution (3.2 mM Na₂HPO₄, 0.5 mM KH₂PO₄, 1.3 mM KCl, 135 mM NaCl (pH 7.4), 0.05% Tween 20). Next, 100 μl of blocking solution (0.2% gelatin in PBS-Tween) is added to each well and incubated for 30 minutes at room temperature. The plates are emptied and then 100 μl of animal sera to be tested is deposited in the wells and incubated for 2 hours at 37° C. The plate is then emptied and then washed three times with 200 μl per well of PBS-Tween solution. 100 μl of a solution containing the second antibody coupled to biotin (PBS-Tween containing 1-10 μg/ml of biotinylated antibody) is added to each well and incubated for 1 hour at 37° C. The plate is then emptied and then washed four times with 200 μl per well of PBS-Tween solution. 100 μl of PBS-Tween containing streptavidin coupled to peroxidase (Sigma) is added according to the manufacturer's recommendations. The plate is then emptied and then washed four times with 200 μl per well of PBS-Tween solution. Finally, the reaction is visualized by adding peroxidase substrate according to the manufacturer's recommendations (example of a developer substrate that can be used: ABTS (Sigma)). The result is read using a plate reader or fluorometer according to the manufacturer's recommendations.

The capture antibody used can be either an immunopurified polyclonal serum against one T. congolense sialidase protein or a mixture of T. congolense sialidase proteins such as TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2, or a monoclonal antibody recognizing an epitope present on one or more of these T. congolense sialidase proteins. The second antibody is a monoclonal antibody different than the capture antibody which recognizes a different epitope of one or more T. congolense sialidase proteins TcoTS-like 1, TcoTS-like 2, TcoTS-like 3, TcoTS-A1, TcoTS-A2, TcoTS-A3, TcoTS-B1, TcoTS-B2, TcoTS-C, TcoTS-D1 and TcoTS-D2.

What is claimed:

1-30. (canceled)

31. A DNA or RNA molecule comprising at least one nucleotide sequence coding for a trans-sialidase-like of an African trypanosome, selected from the sequences SEQ ID NOs: 1-3, a sequence complementary to a sequence selected from one of the sequences SEQ ID NOs: 1-3, a sequence comprising an identity of at least 70% with one of the sequences SEQ ID NOs: 1-3, a fragment of said sequences, or a nucleotide sequence able to hybridize with one of the sequences SEQ ID NOs: 1-3 under stringent hybridization conditions.

32. A protein encoded by the nucleotide sequence of claim 31.

33. A protein comprising a sequence selected from SEQ ID NOs: 4-6, designated TcoTS-like 1, 2 and 3, respectively, or an antigenic peptide fragment of said protein.

34. An expression cassette comprising a DNA molecule of claim 31.

35. A recombinant vector comprising an expression cassette of claim 34.

36. A recombinant host cell comprising a nucleic acid of claim 31.

37. A host cell of claim 36, wherein said cell is of eukaryotic origin, such as notably mammalian cells, insect cells, fungal cells or yeast cells, or said cell is of prokaryotic origin, such as notably E. coli cells or enterobacteria cells.

38. A protein of claim 32 or claim 33, or an antigenic peptide fragment thereof, wherein said protein or fragment exhibits reactivity with the sera of animals infected by an African trypanosome, preferably selected from Trypanosoma congolense, Trypanosoma vivax, Trypanosoma evansi and/or Trypanosoma brucei.

39. A vaccine comprising an effective amount of one or more proteins of claim 32 or 33.

40. A method for preventing and/or treating trypanosomosis or pathogeneses induced by trypanosomosis in non human animals, or for preventing and/or treating trypanosomiasis or pathogeneses induced by trypanosomiasis in humans, comprising administering to said human or non-human subject a vaccine of claim 39.

41. A vaccine of claim 39 for protection against infections by Trypanosoma congolense, Trypanosoma vivax, Trypanosoma evansi and/or Trypanosoma brucei.

42. A method of claim 40, wherein said induced pathogeneses comprise anemia, degradations in general health, weight loss and/or immunosuppression in said animals and/or human.

43. A method of claim 40, wherein said non human animals are selected among bovids, ovids, felids, suids, camelids and/or canids.

44. A vaccine of claim 39, wherein said vaccine is a multivalent vaccine which further comprises one or more antigenic peptides and/or antigenic fragments and/or nucleotide sequences coding for said peptides derived from one or more African trypanosome species, preferably derived from flagellar proteins, sialidases, trans-sialidases, lipases, proteases and/or tubulins.

45. A vaccine of claim 39, which further comprises (i) at least one antiparasitic agent, preferably selected from a trypanocide and/or a nonspecific antiparasitic agent for trypanosomes, (ii) at least one anti-infective agent, preferably selected from β-lactams, fosfomycin, glycopeptides or polypeptides with antibiotic activity, bacitracin, aminoglycosides, macrolides, lincosamides, streptogramins, tetracyclines, phenicols, fusidic acid or quinolones, and/or (iii) at least one symptomatic agent, preferably selected from an anti-anemia agent, a hepatoprotective agent and/or a non-steroidal anti-inflammatory drug.

46. A vaccine of claim 39, which further comprises an adjuvant.

47. A vaccine comprising the vaccine of claim 39 and a vaccine and/or or an antigen directed against theileriosis, anaplasmosis, babesiosis, foot-and-mouth disease, clostridiosis, plague, catarrhal fever, contagious bovine pleuropneumonia (CBPP), blackleg, pasteurellosis and/or sheep pox.

48. A monoclonal or polyclonal antibody which binds a protein or an antigenic peptide fragment of claim 33, and/or is obtained by immunological reaction of a non human animal organism and/or a human with at least one protein or an antigenic peptide fragment of claim 33.

49. A probe for identifying African trypanosome parasites, wherein said probe comprises a nucleotide sequence that enables hybridization with a nucleic acid of claim 31.

50. A method for detecting trypanosomosis in a biological sample, such as the blood of a non human animal and/or a human able to be infected by an African trypanosome, wherein said sample and an antibody of claim 48 are brought together under conditions enabling a possible immunological reaction, and the presence or absence of an immune complex is then detected.

51. A kit for diagnosing trypanosomosis in a biological sample, comprising an antibody of claim 48 or a probe of claim 49.
